SEQUENCE LISTING (21 sequences)

SEQ ID NO: 1 - protein, 8 aa, Artificial
  Artifical sequence derived from human or mouse MUC1 T cell epitope
SAPDTRPA

SEQ ID NO: 2 - protein, 8 aa, Artificial
  Artifical sequence derived from human or mouse MUC1 T cell epitope
SAPDFRPA

SEQ ID NO: 3 - protein, 8 aa, Artificial
  Artifical sequence derived from human or mouse MUC1 T cell epitope
SAPDFRPL

SEQ ID NO: 4 - protein, 8 aa, Artificial
  Artifical sequence derived from human or mouse MUC1 T cell epitope
SAPDTRPL

SEQ ID NO: 5 - protein, 9 aa, Homo sapiens
STAPPAHGV

SEQ ID NO: 6 - protein, 9 aa, Artificial
  Artifical sequence derived from human or mouse MUC1 T cell epitope
TTAPPVHGL

SEQ ID NO: 7 - protein, 8 aa, Artificial
  Artifical sequence derived from human or mouse MUC1 T cell epitope
DLHWASWV

SEQ ID NO: 8 - protein, 9 aa, Homo sapiens
SAPDTRPAP

SEQ ID NO: 9 - protein, 11 aa, Artificial
  Artifical sequence derived from human or mouse MUC1 T cell epitope
XXTAPPXHGXX

SEQ ID NO: 10 - protein, 9 aa, Artificial
  Artifical sequence derived from human or mouse MUC1 T cell epitope
STAPPVHGL

SEQ ID NO: 11 - protein, 9 aa, Artificial
  Artifical sequence derived from human or mouse MUC1 T cell epitope
STAPPAHGL

SEQ ID NO: 12 - protein, 9 aa, Artificial
  Artifical sequence derived from human or mouse MUC1 T cell epitope
TTAPPAHGV

SEQ ID NO: 13 - protein, 9 aa, Artificial
  Artifical sequence derived from human or mouse MUC1 T cell epitope
SAPDTYPAL

SEQ ID NO: 14 - protein, 8 aa, Artificial
  Artifical sequence derived from human or mouse MUC1 T cell epitope
SIINFEKL

SEQ ID NO: 15 - protein, 9 aa, Artificial
  Artifical sequence derived from human or mouse MUC1 T cell epitope
FAPGNYPAL

SEQ ID NO: 16 - protein, 8 aa, Artificial
  Artifical sequence derived from human or mouse MUC1 T cell epitope
RGYVYQGL

SEQ ID NO: 17 - protein, 9 aa, Artificial
  Artificial sequence derived from a MUC1 T cell epitope
SAPDTXRPA

SEQ ID NO: 18 - protein, 8 aa, Artificial
  Artifical sequence derived from human or mouse MUC1 T cell epitope
DAHWESWL

SEQ ID NO: 19 - protein, 8 aa, Artificial
  Artifical sequence derived from human or mouse MUC1 T cell epitope
DAHWRSWL

SEQ ID NO: 20 - protein, 8 aa, Artificial
  Artifical sequence derived from human or mouse MUC1 T cell epitope
DAHWYSWL

SEQ ID NO: 21 - protein, 8 aa, Artificial
  Artifical sequence derived from human or mouse MUC1 T cell epitope
DAHWFSWL

(19) United States
(12) Patent Application Publication
(10) Pub. No.: US 2009/0317414 A1
(43) Pub. Date: Dec. 24, 2009

(54) CANCER VACCINE COMPRISING A MUCIN 1 (MUC1) T CELL EPITOPE-DERIVED PEPTIDE

(75) Inventors: Geoffrey Allan Pietersz, Victoria (AU); Vasso Apostolopoulos, Victoria (AU); Ian Farquhar Campbell McKenzie, Victoria (AU)

Correspondence Address: SHERIDAN ROSS PC, 1560 BROADWAY, SUITE 1200, DENVER, CO, 80202, US

(73) Assignee: 4G VACCINES PTY LTD, Victoria (AU)

(21) Appl. No.: 12/374,591

(22) PCT Filed: Jul. 25, 2007
(86) PCT No.: PCT/AU2007/001031

§ 371 Date: Jan. 21, 2009

(30) Foreign Application Priority Data
Jul. 25, 2006 (AU) 2006904057

(51) Int. Cl. A61K 39/00; C07K 7/06; A61K 35/12

(52) U.S. Cl. 424/185.1; 530/328; 424/93.7

(57) ABSTRACT

A cancer vaccine, and a composition for the ex vivo priming of dendritic cells, is disclosed which comprises a MUC1 T cell epitope-derived peptide or peptide analogue capable of provoking a cytotoxic T cell immune response. Particular MUC1 T cell epitope-derived peptides disclosed include TTAPPVHGL, STAPPVHGL, STAPPAHGL, TTAPPAHGV and SAPDTYPAL.

### FIELD OF THE INVENTION

The present invention relates to the prevention and/or treatment of cancer characterised by Mucin 1-positive (MUC1⁺) tumour cells. More particularly, the present invention relates to a cancer vaccine and composition for the ex vivo priming of dendritic cells, each comprising a MUC1 T cell epitope-derived peptide or peptide analogue.

### INCORPORATION BY REFERENCE

This patent application claims priority from:
AU 2006904057 entitled “A cancer vaccine” filed on 25 Jul. 2006. The entire content of this application is hereby incorporated by reference.

### BACKGROUND OF THE INVENTION

The treatment and/or prevention of cancer is hindered by the complexity of the interactions involved, with tolerance to tumour associated antigens (TAAs) being one significant obstacle.

Mucin 1 (MUC1) is an example of a TAA. The mucins (which include MUC1) are high molecular weight glycoproteins expressed in normal tissues and over-expressed on cancer cells, such as the cells of breast, ovary, colon and pancreatic carcinomas. MUC1 is of interest as a potential target for tumour immunotherapy because (i) there is up to a 100-fold increase in the amount of mucin present on cancer cells compared to normal cells; (ii) MUC1 has a ubiquitous, rather than focal, cellular distribution and (iii) MUC1 has altered glycosylation, revealing peptide epitopes not easily identified in normal mucins.

In mice, it has been previously shown that a 20-mer MUC1 variable number tandem repeat (VNTR) fusion protein conjugated to oxidised mannan (M-FP) generates H2-restricted cytotoxic T lymphocytes (CTLs) which protect mice from challenge against MUC1⁺ mouse tumours [8, 14-21]. In humans, T- and B-cell immune responses to particular epitopes of MUC1 from ovarian, breast, pancreatic and colon cancer patients have been observed [10-12] and circulating immune complexes to MUC1 have been detected in serum of breast and ovarian carcinoma patients [13]. These observations indicate that MUC1 is indeed a suitable target for immunotherapy.

Peptide-based vaccines represent a class of molecules which can be easily synthesised and are devoid of any oncogenic potential. These chemical entities can also be readily modified in order to limit the potential for autoimmune reactions.

The efficacy of peptide immunisation depends on the ability of peptides to induce and activate high avidity CTL. High affinity peptides from non-self antigens that bind to major histocompatability complex (MHC) class I molecules usually induce such high avidity CTLs. Human leukocyte-associated antigen 2 (HLA-A2) is the most common human MHC class I protein in Caucasian populations. HLA-A2 preferentially binds 9-mer peptides with particular anchor residues at positions 2 and 9 and to a lesser extent, position 6. Most non-self CTL epitopes known to-date have been identified because they possess these high-affinity binding (or “canonical”) residues.

However, if the peptide is derived from an over-expressed self tumour antigen, vaccination may not be effective. Since most tumour antigens are self antigens, their specific CTL repertoire would most likely be deleted, as demonstrated by p53 cancer antigen [23-25] leading to tolerance. Because this tolerance is particularly associated with high affinity MHC-associated epitopes, epitopes of lower MHC affinity may therefore represent preferred candidate peptides for tumour immunotherapy [16-22].

Two significant problems exist, however, in the use of lower affinity epitopes.

First, peptide epitopes of lower affinity are unlikely to conform with the predicted epitope motifs and are thus difficult to identify. Therefore, because such low affinity peptides cannot be detected by elution studies and prediction algorithms, the only effective method for their identification is by systematic binding studies and recognition of peptide-MHC (pMHC) by T-cell receptor (TcR).

Secondly, peptide affinity for the MHC and stability of the peptide-MHC complex has been shown to be a significant factor in overall immunogenicity [29, 30]. In order to overcome this problem, many attempts have been made to improve the affinity of peptides for the MHC by replacement of “anchor” residues with the previously determined canonical amino acids [56]. However, while this can result in enhancement of peptide-MHC interactions and reduced likelihood of tolerance, in many cases mutations to the MHC anchor residues have resulted in CTLs which do not recognise the natural counterpart [35, 36, 37]. These results highlight the importance of balance between MHC affinity and receptor cross reactivity required for effective epitope enhancement.

Previous mutation studies with lower-affinity peptide epitopes have concentrated on substituting the known MHC anchor residues with canonical amino acids without making changes to non-anchor residues.

In work leading up to the present invention, low affinity-binding 9-mer MUC1 peptides were identified which induce HLA-A2 restricted CTLs to the MUC1 human breast cancer antigen. Subsequently, the present inventors made substitutions to the various residues of the MUC1 peptide in an attempt to improve the affinity of the peptide for the MHC class I protein and, further, enhance the binding of the pMHC to the TcR. Surprisingly, it was found that even though some of the mutated MUC1 peptide epitopes did not have the canonical Ile/Leu/Val at position 2, they were still able to bind to HLA-A2 and induce CTLs which specifically lyse MUC1⁺ human breast cancer cell line (MCF7) cells.

### SUMMARY OF THE INVENTION

In a first aspect, the present invention provides a vaccine for the prevention and/or treatment of cancer (i.e. a cancer vaccine), said vaccine comprising at least one Mucin 1 (MUC1) T cell epitope-derived peptide or peptide analogue optionally conjugated to at least one helper molecule that binds to human leukocyte antigen (HLA) class II protein, such that the vaccine, upon administration to a subject, elicits a cytotoxic T cell (CTL) response to Mucin 1.

As used herein, the term “Mucin 1 (MUC1) T cell epitope-derived peptide or peptide analogue” refers to a peptide or peptide analogue, derived from a MUC1 T cell epitope, that is capable of provoking a CTL response (i.e. a CD8⁺ T cell response), but does not encompass a peptide consisting of a Mucin 1 amino acid sequence naturally extent in mice and/or humans. For example, for a MUC1 T cell epitope-derived peptide, the peptide will preferably comprise an amino acid sequence corresponding to one that is naturally extent in mice and/or humans (i.e. a native mouse and/or human MUC1 T cell epitope) but modified inasmuch as the amino acid sequence of the peptide incorporates one or more amino acid substitutions. Preferably, said one or more amino acid substitutions are located at one or more of the non-anchor residues of the relevant native MUC1 T cell epitope. Such one or more amino acid substitutions are preferably chosen so as to cause an increase in binding affinity to HLA class I protein (particularly HLA-A2) and/or enhance the binding of the pMHC to the TcR relative to the native MUC1 T cell epitope.

Preferably, the at least one MUC1 T cell epitope-derived peptide or peptide analogue is derived from human MUC1 T cell epitope(s).

The vaccine is preferably capable of eliciting a CTL response to Mucin 1 that is effective in causing lysis of Mucin 1-positive (MUC1⁺) tumour cells.

In a second aspect, the present invention provides a method of prevention and/or treatment of cancer in a subject, said method comprising administering to said subject an effective amount of a vaccine comprising at least one Mucin 1 (MUC1) T cell epitope-derived peptide or peptide analogue optionally conjugated to at least one helper molecule that binds to human leukocyte antigen (HLA) class II protein, such that the vaccine, upon administration to a subject, elicits a cytotoxic T cell (CTL) response to Mucin 1.

In a third aspect, the present invention provides a composition for the ex vivo priming of dendritic cells (DCs), said composition comprising at least one Mucin 1 (MUC1) T cell epitope-derived peptide or peptide analogue optionally conjugated to at least one helper molecule that binds to human leukocyte antigen (HLA) class II protein.

In a fourth aspect, the present invention provides a method of prevention and/or treatment of cancer in a subject, said method comprising the steps of treating dendritic cells (DCs) ex vivo with a composition comprising at least one Mucin 1 (MUC1) T cell epitope-derived peptide or peptide analogue optionally conjugated to at least one helper molecule that binds to human leukocyte antigen (HLA) class II protein, such that the DCs are primed to MUC1, and thereafter administering the primed DCs to said subject.

The primed DCs, upon administration to the subject, elicit a cytotoxic T cell (CTL) response to Mucin 1. Preferably, that CTL response to MUC1 is effective in causing lysis of Mucin 1-positive (MUC1⁺) tumour cells.

### BRIEF DESCRIPTION OF THE ACCOMPANYING FIGURES

FIG. 1 provides the results of flow cytometric analysis of RMA-S cells pulsed with A, MUC1-8 (SAPDTRPA; SEQ ID NO: 1), B, MUC1-8-5F (SAPDFRPA; SEQ ID NO: 2), C, MUC1-8-5F8L (SAPDFRPL; SEQ ID NO: 3) and D, MUC1-8-8L (SAPDTRPL; SEQ ID NO: 4), before incubation with anti-H-2K^{b} specific antibody. Various peptide concentrations were added and labeled as; 10⁻⁴ M, 10⁻⁵ M, 10⁻⁶ M, 10⁻⁷ M, 10⁻⁸ M, 10⁻⁹ M, 10⁻¹⁰ M, and “no peptide”.

FIG. 2 shows the measurement of IFN-γ secreted by T cells by ELISpot assay. A, C57BL/6 mice and B, MUC1×HLA-A2 transgenic mice were immunised with DC pulsed with i, MUC1-8, ii, MUC1-8-5F, iii, MUC1-8-5F8L or iv, MUC1-8-8L peptides. In all immunised mouse groups, specific IFN-γ secreting CD8 T cells are generated which recognise MUC1-8 (▪), MUC1-8-5F (□), MUC1-8-5F8L (▴) or MUC1-8-8L (Δ) peptides. Ovalbumin (OVA8) was used as a negative control and ConA (non-specific stimulus of T cells) was used as an internal positive control. The data are presented as spot forming units (SFU) per 5×10⁵ cells. Experiments were performed at least twice with 3 mice/group.

FIG. 3A, shows a final electron density map of MUC1-8-5F8L. Cα backbone superimposition of B, MUC1-8-5F8L and MUC1-8 and C, MUC1-8-5F8L and OVA8. MUC1-8-5F8L is in yellow, MUC1-8 in pink and OVA8 in cyan. Cα backbone superimposition of OVA8 (cyan) with D, MUC1-8 (crystal structure), E, MUC1-8-5F (model) and F, MUC1-8-8L (model).

FIG. 4 diagrammatically shows the hydrogen bond network within the H-2K^{b} binding groove for the crystal complexes with A, MUC1-8-5F8L, B, MUC1-8 and C, OVA8. Residues from the peptide are labeled P1-P8 while those from the H-2K^{b} molecules are labeled with the amino acid three-letter code and numerical superscripts with dashed lines for H-bonds. Only the binding groove and peptide are shown.

FIG. 5 provides a diagrammatic representation showing the location of water molecules (cyan spheres) within the binding groove of H-2K^{b} for A, MUC1-8-5F8L, B, MUC1-8 and C, OVA8. Residues from the peptide are labeled P1-P8. The binding groove pockets are also indicated. Note that the canonical anchor residues almost completely fill out the C and F pockets while the small non-canonical anchors leave these pockets largely unoccupied.

FIG. 6 provides a graphical representation of CTL responses after immunisation with the mutated peptides. HLA-A2/Kb mice were immunised with STAPPAHGV (SEQ ID NO: 5) (♦), TTAPPVHGL (SEQ ID NO: 6) (◯), DLHWASWV (SEQ ID NO: 7) (▪), each conjugated to KLH and then to oxidised mannan. Oxidised mannan-MUC1 fusion protein (MFP) (+) was used as an internal positive control. MCF7 MUC1 positive tumour cells were labeled with ⁵¹Cr and used as targets in a standard CTL assay.

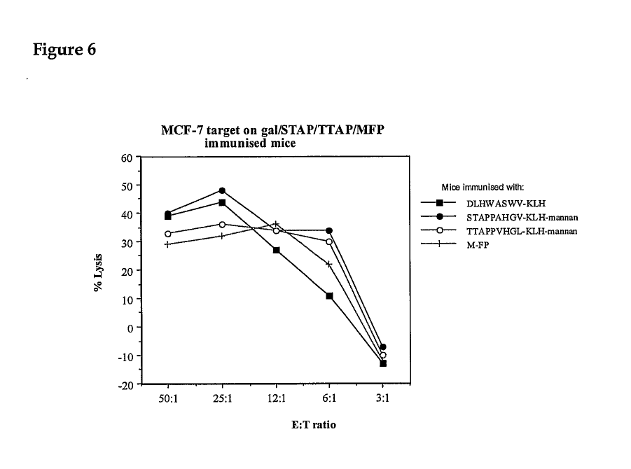

### DETAILED DESCRIPTION OF THE INVENTION

In a first aspect, the present invention provides a vaccine for the prevention and/or treatment of cancer (i.e. a cancer vaccine), said vaccine comprising at least one Mucin 1 (MUC1) T cell epitope-derived peptide or peptide analogue optionally conjugated to at least one helper molecule that binds to human leukocyte antigen (HLA) class II protein, such that the vaccine, upon administration to a subject, elicits a cytotoxic T cell (CTL) response to Mucin 1. As mentioned above, the term “Mucin 1 (MUC1) T cell epitope-derived peptide or peptide analogue” refers to a peptide or peptide analogue, derived from a MUC1 T cell epitope, that is capable of provoking a CTL response, but does not encompass a peptide consisting of a Mucin 1 amino acid sequence naturally extent in mice and/or humans.

Suitable MUC1 T cell epitope-derived peptides preferably comprise an amino acid sequence corresponding to one that is naturally extent in mice and/or humans (i.e. a native mouse and/or human MUC1 T cell epitope) but modified inasmuch as the amino acid sequence of the peptide incorporates one or more amino acid substitutions.

Preferably, said one or more amino acid substitutions are located at one or more of the non-anchor residue positions of the relevant native MUC1 T cell epitope. The non-anchor residues consist of the residues that have not traditionally been recognised by persons skilled in the art as being necessary for high affinity binding of the epitope peptide to MHC/HLA protein. Thus, for example, for high affinity binding of 9-mer peptides to HLA-A2, the anchor residue positions are positions 2 and 9 and to a lesser extent, position 6 (wherein the position numbering conventionally begins with the N-terminal amino acid residue), and the non-anchor residue positions are positions 1, 3, 4, 5, 7 and 8.

Preferably, said one or more amino acid substitutions are also chosen so as to cause an increase in binding affinity to HLA class II protein (particularly HLA-A2) and/or enhance the binding of the pMHC to the TcR relative to the native MUC1 T cell epitope. This can be achieved through substitution of one or more amino acids with amino acids which favour binding of the epitope peptide to HLA class II protein. This may involve substitution of one or more of the amino acids at the anchor residue positions with canonical residues. Thus, for example, to increase the binding affinity of 9-mer peptides having a low or medium binding affinity to HLA-A2, the amino acid at anchor residue position 6 may be substituted with the canonical amino acid Val (V) and/or the amino acid at anchor position 9 may be substituted with the canonical amino acid Leu (L) or Val (V). However, preferably, the amino acid at anchor position 2 is left unchanged (i.e. if the amino acid is non-canonical) or substituted with a non-canonical amino acid such as Thr (T)).

Preferably, the MUC1 T cell epitope-derived peptide is a 9-mer.

As mentioned above, suitable MUC1 T cell epitope-derived peptides are preferably derived from a native mouse and/or human MUC1 T cell epitope (i.e. suitable MUC1 T cell epitope-derived peptides preferably comprise an amino acid sequence corresponding to that of a native mouse and/or human MUC1 T cell epitope but modified by the incorporation of one or more amino acid substitutions). More preferably, suitable MUC1 T cell epitope-derived peptides are derived from a native mouse and/or human MUC1 T cell epitope selected from:

| | |
|---|---|
| (i) STAPPAHGV; | (SEQ ID NO: 5) |
| and | |
| (ii) SAPDTRPAP. | (SEQ ID NO: 8) |

As such, preferred MUC1 T cell epitope-derived peptides include peptides comprising an amino acid sequence corresponding to SEQ ID NO: 5 or SEQ ID NO: 8 but modified by the incorporation of one or more amino acid substitutions (thereby generating a “non-self” epitope), preferably 1 to 4 amino acid substitutions and, more preferably, 2 to 4 amino acid substitutions. The one or more amino acid substitutions may be made at the anchor residue positions, at the non-anchor residue positions, and/or at a combination of anchor and non-anchor residue positions. The one or more amino acid substitutions may also be selected from conservative or non-conservative amino acid substitutions.

Exemplary conservative amino acid substitutions are provided in Table 1 below. Particular conservative amino acid substitutions envisaged are: G, A, V, I, L, M; D, E; N, Q; S, T; K, R, H; F, Y, W, H; and P, Nα-alkylamino acids.

TABLE 1
Exemplary conservative amino acid substitutions

| | Conservative Substitutions |
|---|---|
| Ala | Val*, Leu, Ile |
| Arg | Lys*, Gln, Asn |
| Asn | Gln*, His, Lys, Arg, Asp |
| Asp | Glu*, Asn |
| Cys | Ser |
| Gln | Asn*, His, Lys, |
| Glu | Asp*, γ-carboxyglutamic acid (Gla) |
| Gly | Pro |
| His | Asn, Gln, Lys, Arg* |
| Ile | Leu*, Val, Met, Ala, Phe, norleucine (Nle) |
| Leu | Nle, Ile*, Val, Met, Ala, Phe |
| Lys | Arg*, Gln, Asn, ornithine (Orn) |
| Met | Leu*, Ile, Phe, Nle |
| Phe | Leu*, Val, Ile, Ala |
| Pro | Gly*, hydroxyproline (Hyp), Ser, Thr |
| Ser | Thr |
| Thr | Ser |
| Trp | Tyr |
| Tyr | Trp, Phe*, Thr, Ser |
| Val | Ile, Leu*, Met, Phe, Ala, Nle |

*indicates preferred conservative substitutions

Further, particularly preferred MUC1 T cell epitope-derived peptides include peptides according to formula (I):

| | |
|---|---|
| (I) X^{a}-X¹-TAPP-X⁶-HG-X⁹-X^{b}; | (SEQ ID NO: 9) |

wherein:

X^{a} is absent or any amino acid or sequence of any two to five amino acids,

X¹ is selected from Ser (S) and Thr (T),

X⁶ is selected from Ala (A), Val (V), Leu (L) and Ile (I),

X⁹ is absent or selected from Val (V), Leu (L), Ile (I), Met (M), Phe (F), Ala (A) and Nle, and

X^{b} is absent or any amino acid or sequence of any two to five amino acids;

with the proviso that one or more amino acid substitutions are incorporated.

Preferably, X^{a} is absent, X¹ is selected from S and T, X⁶ is selected from A and V, X⁹ is selected from V and L, and X^{b} is absent.

Moreover, most preferred are the MUC1 T cell epitope-derived peptides consisting of one of the following amino acid sequences:

| | |
|---|---|
| (i) TTAPPVHGL; | (SEQ ID NO: 6) |
| (ii) STAPPVHGL; | (SEQ ID NO: 10) |
| (iii) STAPPAHGL; | (SEQ ID NO: 11) |
| (iv) TTAPPAHGV; | (SEQ ID NO: 12) |
| and | |
| (v) SAPDTYPAL. | (SEQ ID NO: 13) |

Suitable MUC1 T cell epitope-derived peptide analogues include analogues of peptides according to formula (I) and analogues of peptides consisting of one of the amino acid sequences shown above as SEQ ID NO: 5, SEQ ID NO: 6, SEQ ID NO: 8, SEQ ID NO: 10, SEQ ID NO: 11, SEQ ID NO: 12 and SEQ ID NO: 13. Such analogues may be designed using any of the methods well known to persons skilled in the art for designing analogues of peptides based upon peptide sequences in the absence of secondary and tertiary structural information [60]. For example, peptide analogues may be produced by modifying amino acid side chains to increase the hydrophobicity of defined regions of the peptide (e.g. substituting hydrogens with methyl groups on aromatic residues of the peptides), substituting amino acid side chains with non-amino acid side chains (e.g. substituting aromatic residues of the peptides with other aryl groups), and substituting amino- and/or carboxy-termini with various substituents (e.g. substituting aliphatic groups to increase hydrophobicity). Alternatively, suitable MUC1 T cell epitope-derived peptide analogues may be so-called peptoids (i.e. non-peptides) which include modification of the peptide backbone (i.e. introducing amide bond surrogates by, for example, replacing the nitrogen atoms in the peptide backbone with carbon atoms), or include N-substituted glycine residues, one or more D-amino acids (in place of L-amino acid(s)) and/or one or more α-amino acids (in place of β-amino acids or γ-amino acids). Further, suitable MUC1 T cell epitope-derived peptide analogues include “retro-inverso peptides” where the peptide bonds are reversed and D-amino acids assembled in reverse order to the order of the L-amino acids in the peptide sequence upon which they are based, and other non-peptide frameworks such as steroids, saccharides, benzazepine 1,3,4-trisubstituted pyrrolidinone, pyridones and pyridopyrazines.

It is preferred that the vaccine comprise the at least one helper molecule that binds to HLA class II protein; which can be conjugated (either covalently or non-covalently) to the MUC1 T cell epitope-derived peptide or peptide analogue. The presence of the at least one HLA class II protein-binding helper molecule is effective in stimulating helper (CD4⁺) T cells.

The at least one HLA class II protein-binding helper molecule may be any of those well known to persons skilled in the art including, for example, keyhole limpet haemocyanin (KLH), tetanus toxoid (TT), diphtheria toxoid, or smaller T cell helper epitopes such as PADRE peptides [58], and combinations thereof. Preferably, the at least one helper molecule binds to two or more HLA class II protein types or haplotypes. Most preferably, the at least one HLA class II protein-binding helper molecule is KLH.

The at least one HLA class II protein-binding helper molecule is preferably covalently conjugated to the MUC1 T cell epitope-derived peptide or peptide analogue via a short linker(s); for example, a single amino acid or a short amino acid sequence of 2 to 15, more preferably 3 to 10, amino acids in length. An example of a suitable linker is (Lys-Gly)₅.

The vaccine may further comprise a pharmaceutically acceptable carrier. As used herein, the term “pharmaceutically acceptable carrier” is to be understood as referring to any solution, substance or combination thereof that is not biologically or otherwise undesirable (i.e. such that the carrier may be administered to a subject along with the active agent without causing any substantial adverse reaction). Accordingly, the carrier may be selected from excipients and other additives such as diluents (e.g. water, saline, glycerol, ethanol or the like), detergents, colouring agents, wetting or emulsifying agents, pH buffering agents, preservatives and the like, and combinations thereof. Additionally or alternatively, the carrier may comprise any protein, peptide, polypeptide, polysaccharide or other molecule which enhances the ability of an antigen or epitope to be transported intracellularly and thereafter processed and presented onto the cell surface in association with MHC class I or II molecules. Examples of such carrier molecules include mannan, oxidised mannan, partially oxidised mannan, reduced mannan, the TAT protein from human immunodeficiency virus (HIV), the VP22 protein from herpes simplex virus (HSV), the amphipathic peptide Pep-1, the 60 amino acid DNA binding domain (the “homeodomain”) of the Drosophila melanogaster transcription factor, Antennapedia, the 16 amino acid region of Antennapedia responsible for cellular internalisation (i.e. the “penetratin” or “int” peptide) and other receptor-mediated carrier molecules. These carrier molecules may be, for example, covalently conjugated to the MUC1 T cell epitope-derived peptide or peptide analogue, either directly or indirectly (e.g. via a HLA class II protein-binding helper molecule), through a short linker(s) such as those described above. A particularly preferred carrier molecule is oxidised mannan [61]. Preferably, oxidised mannan is covalently conjugated to the MUC1 T cell epitope-derived peptide or peptide analogue via a HLA class II protein-binding helper molecule. The vaccine, upon administration to a subject, elicits a CTL response to MUC1. Preferably, that CTL response to MUC1 is effective in causing lysis of Mucin 1-positive (MUC1⁺) tumour cells.

The vaccine may be used to prevent or treat cancers characterised by MUC1⁺ tumour cells. Such cancers include ovarian, breast, pancreatic and colon cancers.

In a second aspect, the present invention provides a method of prevention and/or treatment of cancer in a subject, said method comprising administering to said subject an effective amount of a vaccine comprising at least one Mucin 1 (MUC1) T cell epitope-derived peptide or peptide analogue optionally conjugated to at least one helper molecule that binds to human leukocyte antigen (HLA) class II protein, such that the vaccine, upon administration to a subject, elicits a cytotoxic T cell (CTL) response to Mucin 1.

As used herein, the term “subject” is to be understood as referring to any animal. However, preferably, the subject is a mammal and, more preferably, selected from the group consisting of humans, livestock such as thoroughbred horses, and companion animals such as dogs and cats. Of course, most preferably, the subject is a human.

The vaccine of the present invention will typically be formulated for administration to a subject as an injectable formulation (e.g. intravenous, intramuscular, intraperitoneal, subcutaneous or intradermal) either as a liquid solution or suspension, however formulations suitable for other routes of administration such as oral, nasal, buccal, vaginal and rectal administration are also contemplated. The vaccine of the present invention will also typically be formulated so as to avoid the use of adjuvants incorporating denaturing agents such as sodium dodecyl sulphate (SDS), however other pharmaceutically acceptable adjuvant systems may be suitable including, for example, aluminium hydroxide, aluminium phosphate, aluminium potassium sulphate (alum) and growth factors and cytokines.

The vaccine of the present invention will be administered to a subject at an effective amount to prevent and/or treat a cancer in a subject. Such an amount may be regarded as being a “therapeutically-effective amount” (i.e. an amount which is effective to elicit a CTL response to MUC1 and, particularly, MUC1⁺ tumour cells). Therefore, as used herein, the terms “effective amount” and “therapeutically effective amount” are to be understood as referring to an amount of the vaccine that is sufficient to provide the desired therapeutic or physiological effect or outcome. As will be appreciated by persons skilled in the art, such an effect or outcome may be accompanied by some undesirable effect or effects (e.g. side effects); hence, a practitioner may need to balance the potential benefits against the potential risks in determining what is an appropriate “effective amount”. Moreover, the exact amount required will vary from subject to subject, depending on the species, age and general condition of the subject, mode of administration and the like. However, by way of example, a subject may be given an effective amount of the vaccine so as to provide a dose of the MUC1 T cell epitope peptide or peptide analogue in the range of 1 to 10,000 μg, more preferably 5 to 500 μg, per administration.

Use of the vaccine of the present invention for the treatment of cancer in a subject may be made in combination with one or more traditional cancer treatments such as radiotherapy, chemotherapy (e.g. using anthracyclines, 5-fluorouracil (5FU), topoisomerase inhibitors, cisplatin and carboplatin), or hormone therapy or therapies utilising hormone modifiers (e.g. catamoxifen). It is to be understood that the present invention extends to such combination therapies.

It is further to be understood that the present invention extends to compositions for ex vivo use (e.g. for dendritic cell therapies for cancer).

Thus, in a third aspect, the present invention provides a composition for the ex vivo priming of dendritic cells (DCs), said composition comprising at least one Mucin 1 (MUC1) T cell epitope-derived peptide or peptide analogue optionally conjugated to at least one helper molecule that binds to human leukocyte antigen (HLA) class II protein.

The at least one MUC1 T cell epitope-derived peptide or peptide analogue may be as described above in relation to the vaccine of the present invention.

Therefore, most preferably, the composition comprises at least one MUC1 T cell epitope-derived peptide consisting of one of the following amino acid sequences:

| | |
|---|---|
| (i) TTAPPVHGL; | (SEQ ID NO: 6) |
| (ii) STAPPVHGL; | (SEQ ID NO: 10) |
| (iii) STAPPAHGL; | (SEQ ID NO: 11) |
| (iv) TTAPPAHGV; | (SEQ ID NO: 12) |
| and | |
| (v) SAPDTYPAL. | (SEQ ID NO: 13) |

The MUC1 T cell epitope-derived peptide or peptide analogue may be conjugated (either covalently or non-covalently) to at least one helper molecule that binds to HLA class II protein (e.g. KLH) and/or a pharmaceutically acceptable carrier as described above. A particularly preferred pharmaceutically acceptable carrier is oxidised mannan.

In a fourth aspect, the present invention provides a method of prevention and/or treatment of cancer in a subject, said method comprising the steps of treating dendritic cells (DCs) ex vivo with a composition comprising at least one Mucin 1 (MUC1) T cell epitope-derived peptide or peptide analogue optionally conjugated to at least one helper molecule that binds to human leukocyte antigen (HLA) class II protein, such that the DCs are primed to MUC1, and thereafter administering the primed DCs to said subject.

The primed DCs, upon administration to the subject, elicit a cytotoxic T cell (CTL) response to Mucin 1. Preferably, that CTL response to MUC1 is effective in causing lysis of Mucin 1-positive (MUC1⁺) tumour cells.

The primed DCs may be used to prevent or treat cancers characterised by MUC1⁺ tumour cells. Such cancers include ovarian, breast, pancreatic and colon cancers.

The step of treating the DCs with the composition ex vivo can be achieved by any of the methods well known to persons skilled in the art such as culturing and/or incubating DCs in the presence of said composition under suitable conditions. Preferably, said step of treating involves the well known technique of “pulsing” the DCs in the presence of said composition.

The DCs treated in this manner are preferably autologous (i.e. obtained from the subject into which the primed DCs are intended to be administered). The DCs may be prepared for the treatment by performing apheresis on the subject's blood (e.g. using an apheresis unit) and thereafter culturing the isolated white blood cells (i.e. lymphocytes, granulocytes and monocytes) under conditions suitable for the generation of DCs [63]. Once DCs have been generated, the DCs can be treated with the composition comprising the MUC1 T cell epitope-derived peptide or peptide analogue, by adding the composition, for example, to the DC culture. Primed DCs may then be tested for maturity and/or purity, optionally stored in, for example, liquid nitrogen, and then, when needed, resuspended in a suitable pharmaceutically acceptable carrier (e.g. saline) for administration to the subject by, preferably, infusion.

In a fifth aspect, the present invention provides a MUC1 T cell epitope-derived peptide consisting of one of the following amino acid sequences:

| | |
|---|---|
| (i) TTAPPVHGL; | (SEQ ID NO: 6) |
| (ii) STAPPVHGL; | (SEQ ID NO: 10) |
| (iii) STAPPAHGL; | (SEQ ID NO: 11) |
| (iv) TTAPPAHGV; | (SEQ ID NO: 12) |
| and | |
| (v) SAPDTYPAL. | (SEQ ID NO: 13) |

in a substantially purified form.

In a sixth aspect, the present invention provides a fusion polypeptide comprising a MUC1 T cell epitope-derived peptide consisting of one of the following amino acid sequences:

| | |
|---|---|
| (i) TTAPPVHGL; | (SEQ ID NO: 6) |
| (ii) STAPPVHGL; | (SEQ ID NO: 10) |
| (iii) STAPPAHGL; | (SEQ ID NO: 11) |
| (iv) TTAPPAHGV; | (SEQ ID NO: 12) |
| and | |
| (v) SAPDTYPAL. | (SEQ ID NO: 13) |

fused to a helper molecule that binds to human leukocyte antigen (HLA) class II protein.

The HLA class II protein-binding helper molecule may be fused to the MUC1 T cell epitope-derived peptide via a short linker(s) such as a single amino acid or a short amino acid sequence of 2 to 15, more preferably 3 to 10, amino acids in length. Preferably, the HLA class II protein-binding helper molecule is/are fused to the MUC1 T cell epitope derived peptide in a manner permitting expression of the fused polypeptide using standard recombinant expression methodologies.

Thus, in a seventh aspect, the present invention provides a polynucleotide molecule comprising a nucleotide sequence encoding a fusion polypeptide according to the sixth aspect.

The polynucleotide molecule may be DNA, RNA or mixtures thereof, and may be in a substantially purified form. The polynucleotide molecule may be included within an expression cassette and/or replicating DNA, RNA or DNA/RNA vectors as are well known to persons skilled in the art (e.g. expression vectors and the like [62]). Such an expression cassette or vector may be introduced into a suitable host cell for expression of the encoded fusion polypeptide by any of the methods well known to persons skilled in the art.

As used herein, the singular forms “a”, “an” and “the” include plural aspects unless the context clearly dictates otherwise. Thus, for example, reference to “a vaccine” includes a single vaccine, as well as two or more vaccines; reference to “an agent” or “a reagent” includes a single agent or reagent, as well as two or more agents or reagents; and so forth.

The invention will now be described with reference to the following non-limiting examples and accompanying figures.

### Examples

The following abbreviations are used in the examples:

| | |
|---|---|
| BSA | Bovine serum albumin |
| CTL | Cytotoxic T lymphocyte |
| CTLp | Cytotoxic T lymphocyte precursor |
| DCs | Dendritic cells |
| ELISA | Enzyme-linked immunosorbent assay |
| ELISPOT | Enzyme linked immunospot |
| E:T | Effector to target cell ratio |
| FACS | Fluorescence activated cell sorter |
| FITC | Fluorescein isothiocyanate |
| HLA | Human leukocyte antigen |
| kDa | Kilodaltons |
| KLH | Keyhole limpet hemocyanin |
| MHC | Major histocombatibility complex |
| mPBS | Mouse phosphate buffered saline |
| MUC1 | Mucin 1 |
| NaCl | Sodium chloride |
| O/N | Overnight |
| OVA | Ovalbumin |
| OxMan | Oxidised mannan |
| PBS | Phosphate buffered saline |
| PI | Propidium iodide |
| RedMan | Reduced mannan |
| RMSD | Root mean square deviation |
| RT | Room temperature |
| SE | Standard error |
| TAA | Tumour-associated antigen |
| TcR | T cell receptor |

### Example 1

### Mouse MHC Class I Binding Studies—Anchor-Modification of Tumour-Associated MUC1-8 Peptide for Enhanced H-2K^{b} Binding

In this example, the non-canonical tumour associated peptide from MUC1, MUC1-8 (SAPDTRPA; SEQ ID NO: 1), was modified at the MHC anchor residues to SAPDFRPL (SEQ ID NO: 3) (MUC1-8-5F8L). This resulted in enhanced binding to H-2K^{b} and improved immune responses. Further, the crystal structure of MUC1-8-5F8L in complex with H-2K^{b} was determined and revealed that binding of the peptide to MHC is similar to that of the canonical peptide OVA8 (SIINFEKL; SEQ ID NO: 13).

Materials and Methods

Peptides

The peptides, SAPDTRPA (SEQ ID NO: 1) (MUC1-8) and the anchor modified analogs SAPDFRPA (SEQ ID NO: 2) (MUC1-8-5F), SAPDFRPL (SEQ ID NO: 3) (MUC1-8-5F8L) and SAPDTRPL (SEQ ID NO: 4) (MUC1-8-8L) were synthesised. The high affinity binding peptides, SIINFEKL (SEQ ID NO: 14)—Chicken ovalbumin₂₅₇₋₂₆₄ (OVA8), FAPGNYPAL (SEQ ID NO: 15)—Sendai virus NP₃₂₄₋₃₃₂ (SEV9) and RGYVYQGL (SEQ ID NO: 16)—Vesicular stomatitis virus NP₅₂₋₅₉ (VSV8) were used to compare to MUC1-8 anchor modified peptides. All peptides were synthesised by Chiron Mimotopes (VIC, Australia) and the purity was >95% and molecular weights were confirmed by electrospray mass spectroscopy.

Production of Soluble H-2K^{b}

The soluble extracellular domains of H-2K^{b} (heavy chain residues 1-274 with C-terminal His-tag and β₂-microglobulin residues 1-99) were expressed in D. melanogaster cells, under the control of a metallothionein promoter as previously described [46, 47, 48]. Briefly, D. melanogaster cells were expanded to large scale (up to 6 L) in serum free Insect Xpress® media (Cambrex Corporation, East Rutherford, N.J., United States of America) and CuSO₄ (625 μM final concentration) was added 3-5 days before harvesting to induce expression of H-2K^{b}. The supernatant was concentrated using a CENTRAMATE tangential flow concentrator (PALL Corporation, East Hills, N.Y., United States of America) using a 10 kDa MWCO membrane (PALL) and then loaded onto an Ni-NTA column and eluted using 10-250 mM imidazole buffer gradient, pH 7.5. Further purification was achieved using a Mono-Q column (GE Healthcare, United States of America; elution using 25-500 mM NaCl gradient, in a Tris-HCl buffer, pH 8.0) and the final sample was dialysed overnight against double distilled water. This was further concentrated using a Nanosep 10 kDa MWCO concentrator (PALL) to achieve final concentrations of 10-15 mg/ml, which where checked using the NanoDrop spectrophotometer (NanoDrop Technologies Inc, Wilmington, Del., United States of America).

Affinity Measurements

Affinity measurements for binding of peptides to soluble H-2K^{b} molecules were performed as previously described [48, 49]. All affinity measurements were repeated at least 2-3 times.

Peptide Stabilisation Assay Using RMA-S Cells

MHC class I molecules in the murine cell line RMA-S (C57BL/6 TAP2-deficient cells) can be used to measure the direct binding of peptides to class I molecules in vitro. RMA-S cells (5×10⁵ cells) were incubated with peptides (10⁻⁴-10⁻¹³ M) at 26° C. for 3 hr then transferred to 37° C. for 30 min. After washing with 0.5% BSA/PBS (2 ml), anti-H-2K^{b} (HB-158) IgG2a as supernatant (1/50 dilution, 100 μl) was added to RMA-S peptide loaded cells and incubated for 45 minutes at 4° C. The cells were washed once again and 100 μl (1:500 dilution) of FITC-conjugated sheep (Fab′)₂ anti-mouse immunoglobulin was added and incubated for 45 min at 4° C.; after further washing, cells were analyzed by FACScan.

Mice, Generation of Dendritic Cells (DC) and Immunisations

C57BL/6 (H-2^{b}) or MUC1×HLA-A2 (H-2^{b}/H-2^{d}/HLA-A2) transgenic 6-8 week old mice were used. Bone marrow cells from C57BL/6 or MUC1×HLA-A2 female mice were cultured at 1×10⁶ cells/ml in tissue culture media, supplemented with 10 ng/ml GM-CSF/IL-4. At day 6, cells were washed, re-suspended in the same culture media and 20 μg/ml peptides were loaded on DCs for 3 hr. Pulsed DC were washed and 100 μl (1-2×10⁶ cells) injected intradermally in female mice into the base of tail. After 14 days mice were boosted and 16 days later splenocytes assessed by ELISpot.

ELISpot Assay

To assess peptide specific IFN-γ production by CD8⁺ T cells to each peptide, splenocytes from immunised mice were used in IFN-γ ELISpot assays. Spleen cells were incubated with 10⁻⁵-10⁻¹⁰ M of each MUC1 peptide, irrelevant peptide (OVA8, SIINFEKL; SEQ ID NO: 8) or internal positive control concanavalin A (ConA) for 18 hr at 37° C., 8% CO₂ on nitrocellulose plates [pre-coated with an anti-murine IFN-γ monoclonal antibody]. Plates were developed as previously described [50].

Preparation and Crystallisation of H-2K^{b}/MUC1-8-5F8L Complex

The soluble extracellular domains of H-2K^{b} (heavy chain residues 1-274 and β₂-microglobulin residues 1-99) were expressed in D. melanogaster cells, as previously described [46, 47, 48]. Large crystals of the H-2K^{b}-MUC1-8-5F8L complex (>10 mg/ml) grew in 1.8-2.0 M NaH₂PO₄/K₂HPO₄ with 1-2% (v/v) 2-methyl-2,4-pentanediol (MPD), pH 6.6-7.4, at 18° C. with 5-fold molar excess of MUC1-8-5F8L peptide. H-2K^{b} and peptide were both in double distilled water (pre-incubated for 3 hrs prior to crystal set up), and crystals were set up using the sitting drop vapor diffusion method, with 0.5 μl MHC-peptide mixture applied to the platform immediately followed by the addition of 0.5 μl of mother liquor. One (1) ml of mother liquor was added to each well of a 24 well Cryschem plate (Hampton Research, Aliso Viejo, Calif., United States of America). Crystals appeared within 5 days and grew to dimensions of 0.2×0.2×0.1 mm within 2 weeks.

Data Collection and Structure Determination

Prior to data collection, crystals were harvested in a NaH₂PO₄/K₂HPO₄ solution, 0.2 M lower than that which the crystals were grown, 1-2% MPD, pH 6.6-7.4 and 25% (v/v) glycerol as cryoprotectant. Crystals were cryo-cooled to −170° C. in a nitrogen gas stream. X-ray diffraction data were collected using a MicroMax007 Rigaku X-ray generator operated at 40 kV and 20 mA. X-rays were focused to 0.3 mm diameter using Osmic Blue confocal optics and diffraction images (Δφ=0.5°) were captured on an R-Axis IV⁺⁺ detector at a crystal-to-detector distance of 200 mm. Diffraction data were processed using the HKL program suite version 1.96.6. Relevant data processing statistics are presented in Table 2.

TABLE 2
Crystallographic data collection and refinement statistics

| Space group | P2₁2₁2 |
|---|---|
| Cell dimensions (Å) | a = 135.26, b = 87.89, |
| | c = 45.16 |
| Resolution range (Å) | 50.0-2.7 (2.8-2.7) |
| R_{merge} (%) | 10.4 (43.4) |
| No. of reflections | 14383 |
| Data completeness (%) | 97.9 (96.2) |
| Data redundancy | 6.4 (5.3) |
| <I/σ(I)> | 17.4 (3.3) |
| R_{work} (%) | 19.3 |
| R_{free} (%) | 24.7 |
| RMSD from ideal values | |
| Bond length (Å) | 0.006 |
| Bond angle (°) | 1.4 |
| Dihedral angle (°) | 24.7 |
| Improper angle (°) | 0.79 |
| B-values from Wilson plot (Å²) (Mean) | 54.4 (37.5) |
| No. residues | |
| Protein | 373 |
| Peptide | 8 |
| No. solvent molecules | |
| H₂O | 104 |
| PO₄³⁻ | 2 |
| MPD | 4 |
| Ramachandran plot values (%) | |
| Most-favoured regions | 89.3 |
| Additionally allowed regions | 9.8 |
| Generously allowed regions | 0.9 |
| Disallowed regions | 0.0 |

The H-2K^{b}-MUC1-8-5F8L crystal structure was determined by phasing the data using the coordinates of the high resolution (1.6 Å) H-2K^{b}-MUC1-8 structure using the CNS program. The MUC1-8 peptide was initially removed and the MUC1-8-5F8L peptide was built into the |Fₒ|−|F_{c}| electron density using the program TURBO-FRODO VERSION 5.5 (BioGraphics, Marseille, France). Cross-validated crystallographic refinements against maximum likelihood targets were carried out with the CNS program suite, version 0.9. Between cycles of crystallographic refinement, the model was fitted to 2|Fₒ|−|F_{c}| and |Fₒ|−|F_{c}| maps on a Silicon Graphics workstation, using the program TURBO-FRODO. The carbohydrate moieties (NAG and FUC), PO4³-ions, solvent (MPD and water) were added to the model when the crystallographic R-factor (R_{work}) dropped below 0.25. A bulk solvent correction and anisotropic overall B-factor refinements were applied during the last cycles of the structure refinement. Table 3 summarises the last stage of refinement and the quality of the model as assessed by PROCHECK (Laskowski). Analysis of the final model with PROCHECK showed 89.3% of the residues are in the most favoured regions of the Ramachandran plot, with none in disallowed regions. The electron density for all residues was well defined, except for the MHC polypeptide turn side chain regions. Figures were prepared using TURBO-FRODO 5.5 and DS Modeling 1.1 (Accelrys Inc, San Diego, Calif., United States of America).

Protein Data Bank Accession Codes

The atomic coordinates and structure factors for the H-2K^{b}-MUC1-8-5F8L complex have been deposited in the RCSB Protein Data Bank with accession code 2F04.

Molecular Modeling

Models of the MUC1-8 peptide analogs (MUC1-8-5F and MUC1-8-8L) were based on the MUC1-8-5F8L crystal structure of the a-chain of the mouse MHC class I molecule H-2K^{b} complexed to MUC1-8-5F8L (described herein). Molecular modeling was performed with the DS Modeling 1.1 software (Accelrys) using the CHARM forcefield to optimise the molecular geometry. Prior to the molecular dynamics simulation, structures were relaxed using steepest descent gradient algorithm until the RMSD was less than 0.1 kcalmol⁻¹, then followed by the Adopted Basis-set Newton-Raphson algorithm until the RMSD was less than 0.01 kcal·mol⁻¹. A distance dependent dielectric was used to simulate aqueous solvent conditions. Peptide molecules and all atoms within a 20 Å radius of the peptide were subsequently heated to RT (300 K) in 1000 steps and equilibrated at this temperature for a further 1000 steps before commencing the molecular dynamics run for 200 ps, storing the structure every 100 steps. The final conformation was selected for all further analysis. The RMSD between the main-chain Cα atoms was calculated for all residues between MUC1-8, OVA8, MUC1-8-5F8L and the anchor-modified analogs (MUC1-8-5F and MUC1-8-8L) as a measure of variation in peptide conformation. All satisfied H-bonds and salt bridges between peptide and H-2K^{b} molecule were identified.

Results

Affinity Measurements of MUC1-8 Peptide Mutants with Soluble H-2K^{b}

As the MUC1-8 peptide did not contain any preferred anchor residues at P2, P5 and P8, it was found to bind with low affinity but induced CTLs in C57BL/6 mice [48]. The affinity of MUC1-8 was measured in an inhibition assay to be 4.3×10⁻⁷ M at 4° C., 8.7×10⁻⁷ M at 23° C. (100-300-fold lower than OVA8, or other high affinity peptides VSV8 and SEV9; Table 3). Mutation of Thr-P5 to Phe-P5 (MUC1-8-5F) increased peptide affinity by at least 7-fold at 23° C. and 37° C., and mutation of Ala-P8 to Leu-P8 (MUC1-8-8L) increased affinity by at least 3-fold at 23° C. and 37° C., whilst the double substitute mutant of Thr-P5/Ala-P8 to Phe-P5/Leu-P8 (MUC1-8-5F8L) increased affinity of the peptide significantly more than the single mutants (i.e. by 14-fold at 23° C.) and provided higher thermal stabilisation at 37° C. (Table 3).

TABLE 3
Affinity measurements of peptides binding to H-2K^{b}

| | | K_{D} | | K_{D} |
|---|---|---|---|---|
| | | 4° C. | K_{D} 23° C. | 37° C. |
| Peptide | Sequence | (nM) | (nM) | (nM) |
| MUC1-8 | SAPDTRPA | 433 | 877 | 37000 |
| | (SEQ ID NO: 1) | | | |
| MUC1-8-5F | SAPDFRPA | 460 | 130 | 4900 |
| | (SEQ ID NO: 2) | | | |
| MUC1-8-8L | SAPDTRPL | 150 | 250 | 8900 |
| | (SEQ ID NO: 4) | | | |
| MUC1-8-5F8L | SAPDFRPL | 160 | 60 | 300 |
| | (SEQ ID NO: 3) | | | |
| OVA8 | SIINFEKL | 6 | 10 | 82 |
| | (SEQ ID NO: 14) | | | |
| VSV8 | RGYVYQGL | 34 | 27 | 163 |
| | (SEQ ID NO: 16) | | | |
| SEV9 | FAPGNYPAL | 5 | 3 | 30 |
| | (SEQ ID NO: 15) | | | |

The affinity values in this table are the means of 5 values [14].
The standard errors of these values are between 10-15%.

Stabilisation of MHC Class I on RMA-S Cells with MUC1-8, MUC1-8-5F, MUC1-8-8L, and MUC1-8-5F8L Peptides

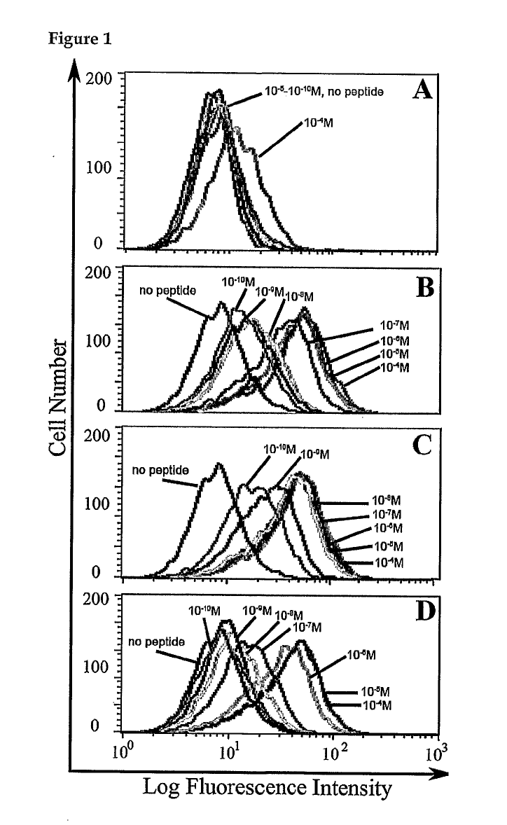

The binding of MUC1-8, MUC1-8-5F, MUC1-8-8L, and MUC1-8-5F8L peptides to MHC class I, H-2K^{b} was determined, in assembly assays, based on peptide dependent stabilisation of MHC heavy chains in TAP2-deficient cells (RMA-S) at 26° C. MUC1-8 stabilised MHC class I, H-2K^{b}, at >10⁻⁴ M; MUC1-8-5F stabilised H-2K^{b}, at >10⁻⁷ M; MUC1-8-8L stabilised H-2K^{b} at >10⁻⁶ M and MUC1-8-5F8L at >10⁻⁹ M (FIG. 1).

Generation of T Cells in vivo

The ex-vivo 18 hour ELISpot assay does not require cell expansion as it detects specifically activated memory effector cells (both CD4 and CD8 cytokine producing terminal effectors). The sensitivity of the assay is higher than limiting dilution analysis, FACscan analysis or ELISA methods and can reliably detect precursor frequencies of antigen specific effectors of 1 in every 500,000 cells [20, 21]. It is therefore an appropriate method to detect antigen specific cells at low precursor frequencies and thus, CTL assays were not performed.

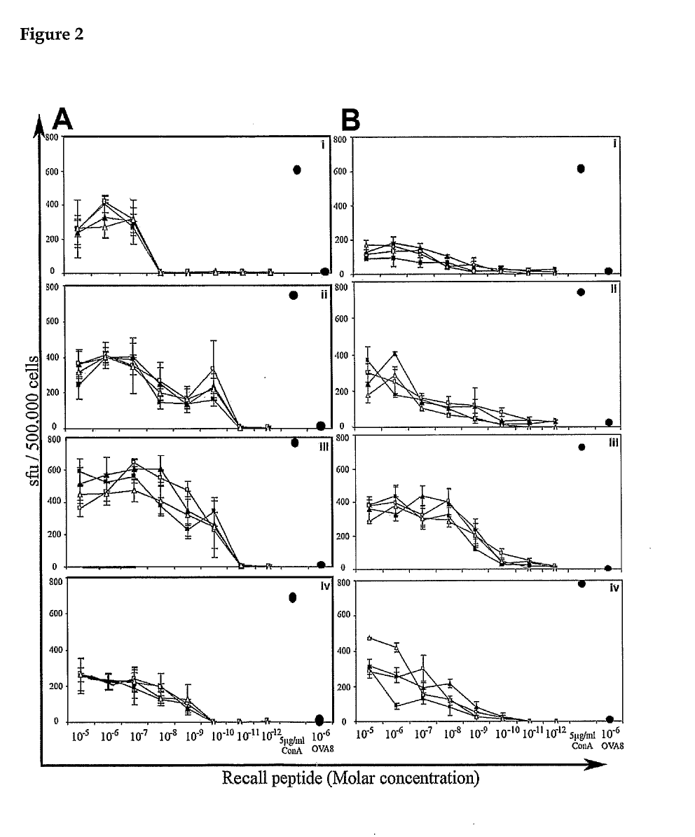

The ability of MUC1-8, MUC1-8-5F, MUC1-8-8L, and MUC1-8-5F8L peptides to induce T cell responses in C57BL/6 mice was measured using IFN-γ by ELISpot analysis after recognition of MUC1-8, MUC1-8-5F, MUC1-8-8L and MUC1-8-5F8L peptides at varying concentrations (10⁻⁵-10⁻¹² M). Mice immunised with DC-MUC1-8 generated IFN-γ secreting T cells which recognised all peptides at a concentration of 10⁻⁷-10⁻⁵ M (FIG. 2A, i). T cells from mice immunised with DC-MUC1-8-5F or MUC1-8-5F8L generated IFN-γ secreting T cells which recognised all peptides in the range of 10⁻¹⁰-10⁻⁵ M, however, the number of sfu/500,000 was higher in mice immunised with MUC1-8-5F8L (FIG. 2A, ii and iii). Mice immunised with MUC1-8-8L generated T cells that recognised all other peptides at 10⁻⁹-10⁻⁵ M (FIG. 2A, iv). Thus, the induction of T cells correlated with the affinity of the peptide, i.e. in increasing strength is, MUC1-8-5F8L>MUC1-8-5F>MUC1-8-8L>MUC1-8.

As it is clear that MUC1-8 is immunogenic in C57BL/6 mice it is not immunogenic in MUC1×HLA-A2 transgenic mice (FIG. 2B, i). However, mutations to the MUC1-8 peptide to result in MUC1-8-5F, MUC1-8-5F8L and MUC1-8-8L increases the magnitude of T cells responses in MUC1×HLA-A2 transgenic mice (where MUC1-8 is self (FIG. 2B); MUC1-8-5F and MUC1-8-8L recognised all peptides 10-fold higher than MUC1-8 (FIG. 2B, i, ii and iv) and mice immunised with double substitute mutant peptide analog MUC1-8-5F8L generated even higher affinity T cells which recognised all peptides (FIG. 2B, iii). Further, immunisation of MUC1-8 peptide yields a precursor frequency of 200 sfu/500,000 cells=1/2,500; immunisation of MUC1-8-5F8L results in a precursor frequency of 400 sfu/500,000 cells=1/1,250. A two fold increase in precursor frequency can have dramatic effects on protective efficacy of peptides in vivo and recognition of tumour cells by CTL. This has been demonstrated for a range of MUC1 antigen formulations, where the precursor frequency correlated with tumour protection and CTL induction, and even a two fold increase gave dramatic effects [53]. In addition, studies in liver-stage malaria have shown a 4 fold increase in protective efficacy of CD8 T cells upon a two fold increase in precursor frequency (from 1/8,000 to 1/4,000) [51]. Importantly, the difference in these frequency values is statistically significant, indicating MUC1-8-5F8L binds with higher affinity, providing quantitative functional data to corroborate the binding assay data demonstrated in FIG. 1 and Table 3. It is clear that, mutations to the native MUC1-8 peptide could overcome tolerance and results in generation of higher affinity T cells.

Binding of MUC1-8-5F8L Mutant Peptide with H-2K^{b}-X-Ray Crystal Structure

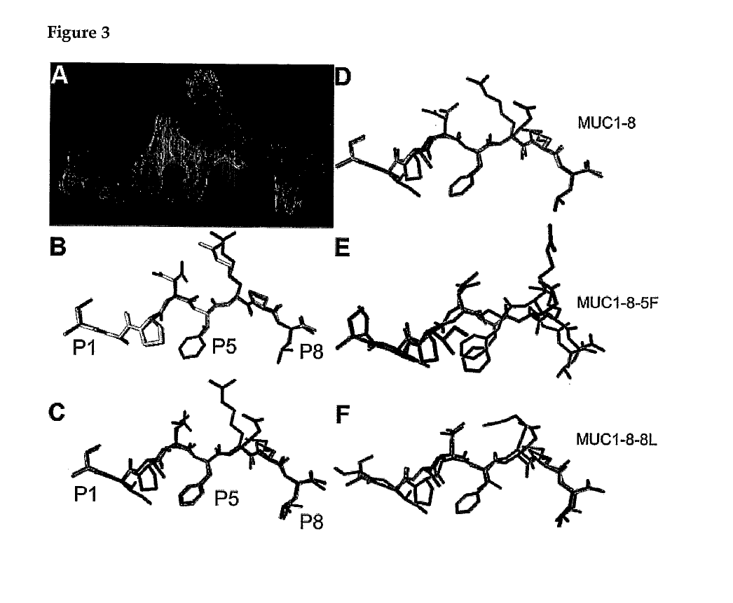

The crystal structure of H-2K^{b}-MUC1-8-5F8L at 2.7 Å resolution was determined by molecular replacement and refined to a final R_{work} of 19.3% and an R_{free} of 24.7% (Table 2). The final atomic coordinates consisted of H-2K^{b} heavy chain (residues 1-274), β₂-microglobulin (residues 1-99), 4 carbohydrate moieties (NAG at Asn⁸⁶ and NAG and FUC at Asn¹⁷⁶ of the heavy chain) and all peptide residues, P1-P8 (MUC1-8-5F8L). Additionally, two phosphate (PO₄³⁻) and four MPD molecules were located. Electron density for the bound peptide was continuous and well resolved (FIG. 3A).

Superposition of MUC1-8-5F8L, MUC1-8 and OVA8 showed similar overlays when viewed from the side (FIGS. 3, B and C). The RMSD between MUC1-8 and MUC1-8-5F8L was very low (0.19 Å, Table 4), while that of OVA8 and MUC1-8-5F8L was considerably higher (0.57 Å, Table 4) but comparable with that between MUC1-8 and OVA8 (RMSD 0.51 Å; FIG. 3D).

TABLE 4
Ca backbone atom RMSD (Å)

| | OVA8 | MUC1-8 | 5F8L |
|---|---|---|---|
| OVA8 | | | |
| MUC1-8 | 0.51 | | |
| 5F8L | 0.57 | 0.19 | |
| 5F | 1.27 | 1.18 | 1.14 |
| 8L | 0.60 | 0.58 | 0.55 |

Interactions of MUC1-8-5F8L Peptide with H-2K^{b}—X-Ray Crystal Structure

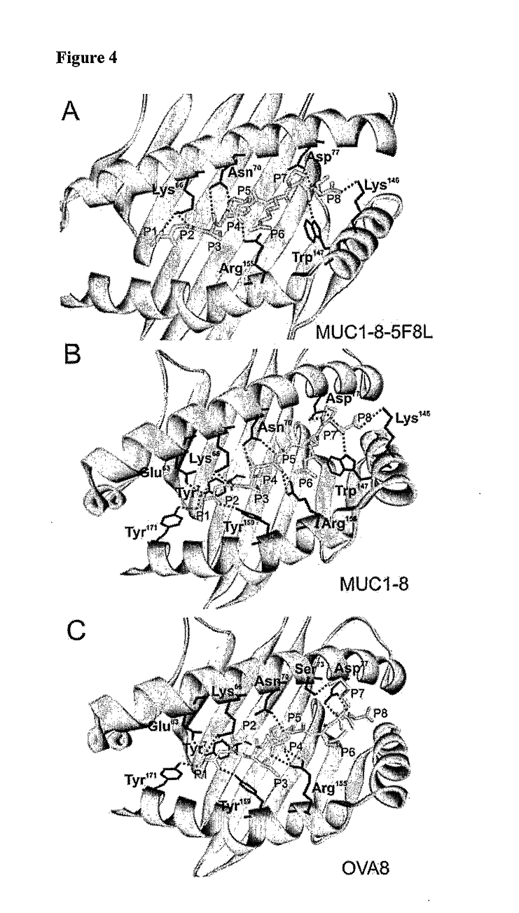

High affinity interactions are consistent with the formation of a highly conserved hydrogen bond network between side-chains of the MHC and the peptide backbone, mainly around the N and C termini, and the optimal fit of peptide side-chains into the MHC pockets. Intermolecular H-bonds between peptide residues and MHC are summarised in Table 5 and FIG. 4.

TABLE 5
Intermolecular H-bonding between H-2K^{b} andpeptides OVA8,
MUC1-8, MUC1-8-5F8L (crystal structures) and MUC1-8-5F and
MUC1-8-8L (models)

| | OVA8 | MUC1-8 | MUC1-8-5F8L | MUC1-8-5F | MUC1-8-8L |
|---|---|---|---|---|---|
| P1 | S^{P1}Oγ:K⁶⁶Nζ | S^{P1}Oγ:K⁶⁶Nζ | S^{P1}Oγ:K⁶⁶Nζ | S^{P1}Oγ:K⁶⁶Nζ | S^{P1}Oγ:K⁶⁶Nζ |
| | S^{P1}O:Y¹⁵⁹OH | S^{P1}O:Y¹⁵⁹OH | | | |
| | S^{P1}N:Y¹⁷¹OH | S^{P1}N:Y¹⁷¹OH | | | |
| | S^{P1}N:Y⁷OH | S^{P1}N:Y⁷OH | | | |
| | S^{P1}Oγ:E⁶³Oε2 | S^{P1}Oγ:E⁶³Oε2 | | | |
| P2 | I^{P2}O:K⁶⁶Nζ | A^{P2}O:K⁶⁶Nζ | A^{P2}O:K⁶⁶Nζ | A^{P2}O:K⁶⁶Nζ | A^{P2}O:K⁶⁶Nζ |
| P3 | I^{P3}O:N⁷⁰Nδ2 | P^{P3}O:N⁷⁰Nδ2 | P^{P3}O:N⁷⁰Nδ2 | P^{P3}O:N⁷⁰Nδ2 | P^{P3}O:N⁷⁰Nδ2 |
| P4 | N^{P4}O:R¹⁵⁵NH1 | D^{P4}O:R¹⁵⁵NH1 | D^{P4}O:R¹⁵⁵NH1 | D^{P4}O:R¹⁵⁵NH1 | D^{P4}O:R¹⁵⁵NH1 |
| | N^{P4}O:R¹⁵⁵NH2 | D^{P4}O:R¹⁵⁵NH2 | D^{P4}O:R¹⁵⁵NH2 | D^{P4}O:R¹⁵⁵NH2 | D^{P4}O:R¹⁵⁵NH2 |
| | N^{P4}Oδ1:R¹⁵⁵NH1 | | | | |
| P5 | F^{P5}N:N⁷⁰Oδ1 | T^{P5}N:N⁷⁰Oδ1 | F^{P5}N:N⁷⁰Oδ1 | F^{P5}N:N⁷⁰Oδ1 | T^{P5}N:N⁷⁰Oδ1 |
| P6 | | | | | |
| P7 | K^{P7}Nζ:S⁷³Oγ | P^{P7}O:W¹⁴⁷Nε1 | P^{P7}O:W¹⁴⁷Nε1 | P^{P7}O:W¹⁴⁷Nε1 | P^{P7}O:W¹⁴⁷Nε1 |
| P8 | L^{P8}N:D⁷⁷Oδ1 | A^{P8}N:D⁷⁷Oδ1 | L^{P8}N:D⁷⁷Oδ1 | A^{P8}N:D⁷⁷Oδ1 | L^{P8}N:D⁷⁷Oδ1 |
| | | A^{P8}N:K¹⁴⁶Nζ | L^{P8}O:K¹⁴⁶Nζ | A^{P8}O:K¹⁴⁶Nζ | L^{P8}O:K¹⁴⁶Nζ |

Note:
Anchor modified residues in bold for peptide MUC1-8-5F8L (crystal structure), MUC1-8-5F and MUC1-8-8L (models).

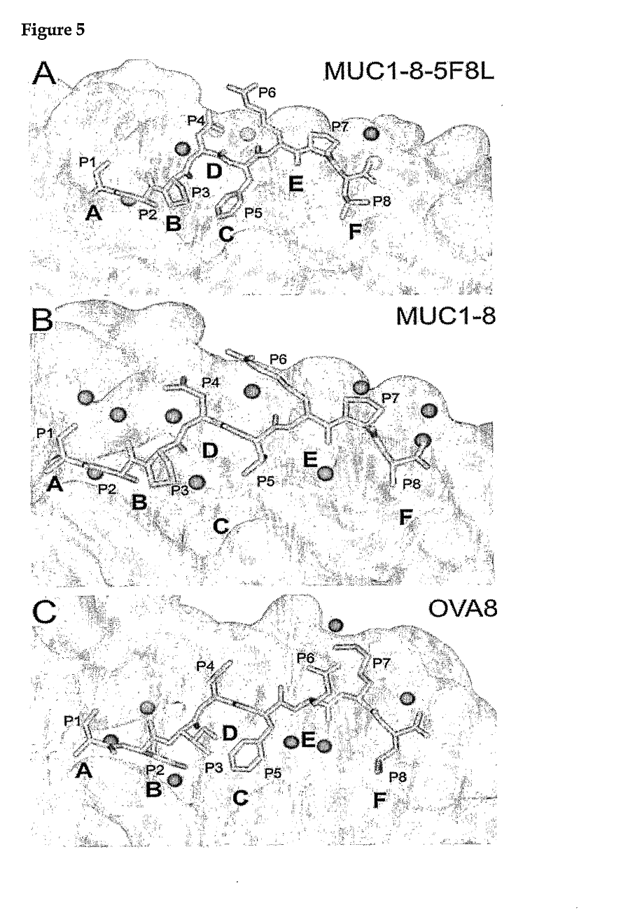

The number of water molecules located in the near vicinity of the peptide within the MHC groove varied between the crystal structures of MUC1-8-5F8L, MUC1-8 and OVA8. Fewer water molecules were found in the MUC1-8-5F8L crystal structure (6 water molecules; FIG. 5A) than is the MUC1-8 crystal structure (10 water molecules; FIG. 5B), or in OVA8 (7 water molecules; FIG. 5C). The intermolecular salt bridges between Asp^{P4} with Arg¹⁵⁵ and Arg^{P6} with Glu¹⁵² were conserved for the MUC1-8-5F8L crystal structure, despite the loss of the salt bridge between Asp^{P4} and Lys⁶⁶, observed for MUC1-8. The intramolecular salt bridge observed between Asp^{P4} and Arg^{P6} was conserved for the MUC1-8-5F8L peptide as was observed for the parent non-canonical peptide MUC1-8. For comparison, intermolecular salt bridges were observed between Arg¹⁵⁵ with Glu^{P6} and Asp⁷⁷ with Lys^{P7} for OVA8, with an intramolecular salt bridge also being present between Glu^{P6} and Lys^{P7}.

Peptide binding within the H-2K^{b} groove is predominantly attributed to the contribution of H-bonding and salt bridge formation. In addition, the involvement of water molecules, which assist with binding of peptide residues to relevant residues within the H-2K^{b} pockets, namely C and F, is also a major contributing factor. Both C and F pockets of the H-2K^{b}-MUC1-8-5F8L crystal are occupied by the P5 and P8 side chains, similar to that observed for OVA8. The modification of the small P5 and P8 anchor residues of MUC1-8 to Phe and Leu, respectively, contributed to the strong binding of the MUC1-8-5F8L peptide to H-2K^{b}. In contrast, the weak binding of MUC1-8 to H-2K^{b} can be attributed to the lack of appropriate anchors at these positions to fill these pockets. As a result, the binding of peptide to MHC is via indirect H-bonding with water molecules in the vicinity of pockets C and F.

Interactions of MUC1-8 Peptide Analogs with H-2K^{b}—Molecular Modeling

The crystal structure of MUC1-8-5F8L was used to model the MUC1-8-5F and MUC1-8-8L peptide analogs. These resulting models were compared with the MUC1-8-5F8L crystal structure (described herein) as well as the parent non-canonical peptide MUC1-8 (1G7Q) and the canonical peptide OVA8 (1VAC) crystal structures. Comparisons were also made between MUC1-8-5F8L, MUC1-8 and OVA8 to assess the effect of anchor modification on binding to H-2K^{b}. RMSDs were calculated for the Cα backbone atoms and are listed in Table 4. Superimposition of MUC1-8-5F8L with the molecular models, MUC1-8-5F and MUC1-8-8L, yielded RMSD values of 1.18 Å and 0.58 Å, respectively. For comparison, all peptides were also superimposed with the canonical OVA8 peptide, which yielded RMSD values of 0.51 Å (MUC1-8), 0.57 Å (MUC1-8-5F8L), 0.60 Å (MUC1-8-8L) and 1.27 Å (MUC1-8-5F) (FIG. 3D-F). The largest deviation was observed between the 5F anchor modified peptide (MUC1-8-5F) with RMSD values of 1.27 Å (with OVA8), 1.18 Å (with MUC1-8) and 1.14 Å (with MUC1-8-5F8L). This data correlated well with the affinity measurements performed at 4° C. (Table 2). The intermolecular H-bonds are summarised in Table 5.

Discussion

The selection of suitable antigens to elicit an effective immunological response in cancer patients is the first step to designing an effective vaccine. However, tolerance to these antigens remains an obstacle to overcome. Further, identification of the antigenic portion and most importantly the immunogenic fragments remains challenging. To date, medium-to-high affinity peptides selected for cancer immunotherapy have resulted in only limited success in clinical trials. For example, partial or complete tumour regression was observed in only 10-30% of patients receiving melanoma peptides [54]. The efficacy of high affinity “self” epitope to induce an effective CTL response and provide protection and eradication of tumour cells has been unsuccessful, due to the absence of T cells, which have been deleted during development of the immune system [23-26]. More recently, low-to-medium affinity peptides have been selected which have yielded more promising results. For example, high vaccination efficiency Was observed for heteroclitic variants of low affinity epitopes from the naturally expressed murine telomerase reverse transcriptase (mTERT) and mice immunised with the low affinity p572 and p988 epitopes resulted in protection against tumour challenge [55].

Previously, the crystal structure of the low affinity non-canonical MUC1-8 peptide in complex with H-2K^{b} was reported [20]. Further, MUC1-8 was also shown to be capable of inducing immune responses in mice despite the low affinity for H-2K^{b}. Herein, the crystal structure of the MUC1-8-5F8L peptide in complex with H-2K^{b} has been reported, which provides insight into the enhanced binding of the parent MUC1-8 peptide to H-2K^{b} following modification of the non-canonical anchor residues to canonical ones (Thr^{P5} to Phe and Ala^{P8} to Leu), MUC1-8-5F8L. Enhanced T cell responses were observed, which varied depending on which anchor was modified. The biological data revealed that modification of the central anchor P5 to Phe (MUC1-8-5F) increased peptide affinity (as measured by competition studies) by 7-fold compared to modification of the P8 residue to Leu (MUC1-8-8L) which only increased peptide affinity 3-fold compared to MUC1-8. In comparison, MUC1-8-5F8L showed a significant 14-fold increase in binding affinity. Similar results were also obtained for the stabilisation studies using TAP2-deficient RMA-S cells as determined by FACS.

T cell responses in vivo also revealed that C57BL/6 mice immunised with the doubly substituted MUC1-8 peptide, MUC1-8-5F8L, recognised all other peptides (MUC1-8, MUC1-8-5F and MUC1-8-8L) more strongly. Although weaker T cell responses were observed in MUC1×HLA-A2 transgenic mice, the overall trend revealed that they are more immunogenic (tolerance could be overcome) with anchor substitutions to the non-canonical low affinity MUC1-8 peptide. Enhanced immunogenicity and binding of non-immunogenic low affinity peptides to HLA-A2.1 has been achieved with a tyrosine substitution at the P1 position. Binding affinity can be increased 55-fold and/or stabilised for more than 2 hours compared to parent peptides. Most importantly, the mutants could trigger CTL which also recognise the parent peptide [42]. Similarly, enhanced immune responses and binding to H-2K^{b} were observed when the low affinity MUT1 peptide, a TAA isolated from 3LL Lewis lung carcinoma, was modified at the anchor positions P3, P5 and P8 [43]. Moreover, mice were immunised with a higher affinity peptide (SAPDTRPA (SEQ ID NO: 1) to SAPDT-GalNAc-RPA (SEQ ID NO: 17)) where the affinity is similar to MUC1-8-5F8L. The T cells that are generated recognised the mutated-higher affinity peptide more efficiently compared to the non-mutated, lower affinity peptide [21].

The crystal structure of MUC1-8-5F8L revealed that a number of H-bonds were lost at the N-terminus, namely Ser^{P1} with Tyr⁷, Glu⁶³, Tyr¹⁵⁹ and Tyr¹⁷¹, when compared to both the crystal structures of MUC1-8 and OVA8 in complex with H-2K^{b}. Different C-terminal H-bonds were also noted (Pro^{P7} with Tyr¹⁴⁷ not Ser⁷³ as for OVA8) and Leu^{P8} with Lys¹⁴⁶ (also observed for MUC1-8 with P8 residue Ala). The bound conformation of the MUC1-8-5F8L peptide was very similar to MUC1-8 main-chain and comparable to that of OVA8. Interestingly, the number of water molecules surrounding the MUC1-8-5F8L peptide was lower (6) than that for MUC1-8 (10) and more comparable to that of OVA8 (7). The C and F pockets are fully occupied by the larger hydrophobic residues, Phe and Leu, and help to stabilise the peptide within the binding groove in a similar manner to OVA8, attributing to the strong binding affinity of this peptide for H-2K^{b}. The large cavities present at both the C and F pockets in the MUC1-8 structure, are occupied with water molecules which help stabilise the low affinity MUC1-8 peptide; these water molecules are absent in the high affinity binding peptides MUC1-8-5F8L and OVA8. It is noteworthy, that compared to OVA8 and MUC1-8, there are no water molecules in the E pocket of H-2K^{b} for the H-2K^{b}-MUC1-8-5F8L crystal structure.

The molecular modeling studies suggested that MUC1-8-5F deviated by the greatest amount from the parent MUC1-8 peptide structure, OVA8 and MUC1-8-5F8L (Table 4). RMSDs between MUC1-8-8L and MUC1-8, OVA8 and MUC1-8-5F8L were lower (Table 4) suggesting that modifications in the C-terminus of the peptide do not affect the peptide-H-2K^{b} complex structure to the same extent; the data correlated well with the affinity data obtained at 4° C.

Overall, the work of this example validated that anchor modifications made to non-canonical tumour epitopes, such as MUC1-8, can significantly enhance binding to the MHC class I molecule H-2K^{b} and, in this case, also improve T cell responses.

### Example 2

### Human Studies—Rationale for Peptide Modifications to Improve Affinity for HLA-A2

MUC1 epitope peptides were identified as previously described [22], and peptides selected that were able to bind with low affinity to HLA-A*0201 class I molecules, but still elicit CTLs which can directly lyse MUC1⁺ human breast cancer cells. Since peptide immunogenicity as determined by in vivo CD8⁺ and CD4⁺ T cell responses had been shown to correlate with peptide binding affinity for MHC class I or II [29, 30], this example investigated whether replacement of “anchor” residues with the previously determined canonical amino acids [31] could result in enhancement of peptide MHC interactions and subsequently also improve immunogenicity.

Previous studies [59] have resulted in the following observations.

Mutations to the Mimic Peptide DAHWESWL (SEQ ID NO: 18)

The mouse mimic peptide (DAHWESWL; SEQ ID NO: 18) does not contain the known HLA-A2 anchor amino acids. A number of mutations to the mouse mimic peptide were made; some produced enhanced immune responses to MUC1, whereas others produced reduced or no specific immune response. That is:

DAHWESWL (SEQ ID NO: 18) generated a CTLp of
1/18,000;
DAHWRSWL (SEQ ID NO: 19) generated a CTLp of
<1/1,000,000;
DAHWYSWL (SEQ ID NO: 20) generated a CTLp of
1/800,000; and
DAHWFSWL (SEQ ID NO: 21) generated a CTLp of
1/130,000.

Thus, mutations to the peptide do not necessarily result in generation of enhanced immune responses.

DAHWESWL (SEQ ID NO: 18) was also mutated to DLHWASWV (SEQ ID NO: 7), in which case certain anchor residues have been substituted to produce a more HLA-A2 compatible peptide. Immunisation of HLA-A2 transgenic mice with peptide DLHWASWV-KLH-oxidised mannan resulted in the generation of CTL that were able to specifically lyse MCF7 cells (human MUC1⁺ breast cancer cell line). This mutated peptide was used as a control in the current experiments.

The low affinity MUC1 peptide (SAPDTRPA; SEQ ID NO: 1) and the low-medium affinity (STAPPAHGV; SEQ ID NO: 5) peptide which bind to HLA-A2 do not contain the canonical anchor amino acids to enable high affinity binding. Residues at P2, P6, P9 point “down” into the peptide binding groove of HLA-A2 [20]. Preferred residues for binding are Leu/Met at P2 and Leu/Val at P9. In addition, Val at P6 acts as auxiliary anchor residue [31]. The remaining amino acid residues are primarily involved in presentation to the TcR.

STAPPAHGV (SEQ ID NO: 5) was converted by mutation (presumably to high affinity) at positions P6 and P9. Two amino acids were substituted, producing the peptide, STAPPVHGL (SEQ ID NO: 10). Further, residue Ser-P1 was mutated to Thr (TTAPPVHGL; SEQ ID NO: 6). Immunisation of HLA-A2 transgenic mice with peptide TTAPPVHGL-KLH-oxidised mannan resulted in the generation of CTLs that were able to specifically lyse MCF7 cells (human MUC1⁺ breast cancer cell line). TTAPPVHGL (SEQ ID NO: 6) lysed MCF7 cells similarly to STAPPAHGV (SEQ ID NO: 5) immunised mice; the lysis was also similar to mannan-MUC1 fusion protein and mimic mutant peptide (DLHWASWV; SEQ ID NO: 7) (FIG. 1).

### Example 3

### Human Studies—Preparation of Conjugates and Immunisation of Mice

Material and Methods

Peptide-KLH Conjugation

Peptide was coupled to KLH via glutaraldehyde. In brief, 2 mg KLH in 1 ml phosphate buffer saline (PBS) and 2 mg peptide in 1 ml PBS were mixed and added dropwise to 1 ml of 0.75% glutaraldehyde, and reacted overnight at room temperature. Glutaraldehyde is a bi-functional coupling reagent that links two compounds through their amino groups.

Oxidised Mannan Conjugation to Peptide-KLH

14 mg mannan was dissolved in 1 ml sodium phosphate buffer (pH 6), followed by the addition of 100 μl 0.1 M sodium periodate (dissolved in pH 6 phosphate buffer) and incubated on ice for 1 hour in the dark. 10 μL ethanediol was then added to the mixture and incubated for a further 30 mins on ice. The resultant mixture (oxidised mannan) was passed through a PD-10 column (Sephadex G-25 M column) pre-equilibrated in pH 9.0 phosphate buffer, to exclude out sodium periodate and ethanediol. Oxidised mannan (7 mg/ml) was eluted with 2 ml of pH 9 phosphate buffer, to which 1 mg peptide-KLH was added and allowed to react overnight at room temperature in the dark. Conjugation occurs via Schiff base formation between free amino groups of KLH and oxidised mannan. Samples were used without further purification. Oxidised mannan was conjugated to MUC1 fusion protein (M-FP) as previously described [8, 15,16, 18, 19, 22, 28].

Immunisation of Mice

HLA-A2/K^{b} transgenic mice were immunised with 5 μg of peptide intraperitoneally on days 0, 10, 17 and 7-10 days later CTL responses were measured using standard CTL assay as previously described [18, 19, 22, 28].

Results and Discussion

The results demonstrated that immunisation of HLA-A2 transgenic mice with peptide TTAPPVHGL (SEQ ID NO: 6)-KLH-oxidised mannan resulted in the generation of CTLs that were able to specifically lyse MCF7 cells (human MUC1⁺ breast cancer cell line). This was surprising, since the mutated epitope did not have the canonical Ile/Leu/Val at position 2.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

All scientific citations, patents, patent applications and manufacturer's technical specifications referred to hereinafter are incorporated herein by reference in their entirety.

Reference to any prior art in this specification is not, and should not be taken as, an acknowledgment or any form of suggestion that that prior art forms part of the common general knowledge in any country.

It is to be understood that unless otherwise indicated, the subject invention is not limited to specific formulation components, manufacturing methods, biological materials or reagents, dosage regimens and the like, as such may vary. It is also to be understood that the terminology used herein is for the purpose of describing particular embodiments only and is not intended to be limiting.

### REFERENCES

1. Gendler S, Taylor-Papadimitriou J, Duhig T, Rothbard J, Burchell J: A highly immunogenic region of a human polymorphic epithelial mucin expressed by carcinomas is made up of tandem repeats. J Biol Chem 1988, 263(26):12820-12823.

2. Xing P X, Reynolds K, Tjandra J J, Tang X L, McKenzie I F: Synthetic peptides reactive with anti-human milk fat globule membrane monoclonal antibodies. Cancer Res 1990, 50(1):89-96.

3. Xing P X, Tjandra J J, Stacker S A, Teh J G, Thompson C H, McLaughlin P J, McKenzie I F: Monoclonal antibodies reactive with mucin expressed in breast cancer. Immunol Cell Biol 1989, 67 (Pt 3):183-195.

4. Barnd D L, Lan M S, Metzgar R S, Finn O J: Specific, major histocompatibility complex-unrestricted recognition of tumour-associated mucins by human cytotoxic T cells. Proc Natl Acad Sci USA 1989, 86(18):7159-7163.

5. Ioannides C G, Fisk B, Jerome K R, Irimura T, Wharton J T, Finn O J: Cytotoxic T cells from ovarian malignant tumours can recognise polymorphic epithelial mucin core peptides. J Immunol 1993, 151(7):3693-3703.

6. Jerome K R, Domenech N, Finn O J: Tumour-specific cytotoxic T cell clones from patients with breast and pancreatic adenocarcinoma recognise EBV-immortalized B cells transfected with polymorphic epithelial mucin complementary DNA. J Immunol 1993, 151(3):1654-1662.

7. Agrawal B, Reddish M A, Longenecker B M: In vitro induction of MUC1 peptide-specific type 1 T lymphocyte and cytotoxic T lymphocyte responses from healthy multiparous donors. J Immunol 1996, 157(5):2089-2095.

8. Apostolopoulos V, Loveland B E, Pietersz G A, McKenzie I F: CTL in mice immunised with human Mucin 1 are MHC-restricted. J Immunol 1995, 155(11):5089-5094.

9. Domenech N, Henderson R A, Finn O J: Identification of an HLA-A11-restricted epitope from the tandem repeat domain of the epithelial tumour antigen mucin. J Immunol 1995, 155(10):4766-4774.

10. Kotera Y, Fontenot J D, Pecher G, Metzgar R S, Finn O J: Humoral immunity against a tandem repeat epitope of human mucin MUC1 in sera from breast, pancreatic, and colon cancer patients. Cancer Res 1994, 54(11):2856-2860.

11. Rughetti A, Turchi V, Ghetti C A, Scambia G, Panici P B, Roncucci G, Mancuso S, Frati L, Nuti M: Human B-cell immune response to the polymorphic epithelial mucin. Cancer Res 1993, 53(11):2457-2459.

12. von Mensdorff-Pouilly S, Gourevitch M M, Kenemans P, Verstraeten A A, Litvinov S V, van Kamp G J, Meijer S, Vermorken J, Hilgers J: Humoral immune response to polymorphic epithelial mucin (MUC1) in patients with benign and malignant breast tumours. Eur J Cancer 1996, 32A(8):1325-1331.

13. Gourevitch M M, von Mensdorff-Pouilly S, Litvinov S V, Kenemans P, van Kamp G J, Verstraeten A A, Hilgers J: Polymorphic epithelial mucin (MUC1)-containing circulating immune complexes in carcinoma patients. Br J Cancer 1995, 72(4):934-938.

14. Acres B, Apostolopoulos V, Balloul J M, Wreschner D, Xing P X, Ali-Hadji D, Bizouarne N, Kieny M P, McKenzie I F: MUC1-specific immune responses in human MUC1 transgenic mice immunised with various human MUC1 vaccines. Cancer Immunol Immunother 2000, 48(10):588-594.

15. Apostolopoulos V, Barnes N, Pietersz G A, McKenzie I F: Ex vivo targeting of the macrophage mannose receptor generates anti-tumour CTL responses. Vaccine 2000, 18(27):3174-3184.

16. Apostolopoulos V, Haurum J S, McKenzie I F: MUC1 peptide epitopes associated with five different H-2 class I molecules. Eur J Immunol 1997, 27(10):2579-2587.

17. Apostolopoulos V, Pietersz G A, Gordon S, Martinez-Pomares L, McKenzie I F: Aldehyde-mannan antigen complexes target the MHC class I antigen-presentation pathway. Eur J Immunol 2000, 30(6):1714-1723.

18. Apostolopoulos V, Pietersz G A, Loveland B E, Sandrin M S, McKenzie I F: Oxidative/reductive conjugation of mannan to antigen selects for T1 or T2 immune responses. Proc Natl Acad Sci USA 1995, 92(22):10128-10132.

19. Apostolopoulos V, Pietersz G A, McKenzie I F: Cell-mediated immune responses to MUC1 fusion protein coupled to mannan. Vaccine 1996, 14(9):930-938.

20. Apostolopoulos V, Yu M, Corper A L, Teyton L, Pietersz G A, McKenzie I F, Wilson I A, Plebanski M: Crystal structure of a non-canonical low-affinity peptide complexed with MHC class I: a new approach for vaccine design. J Mol Biol 2002, 318(5):1293-1305.

21. Apostolopoulos V, Yuriev E, Ramsland P A, Halton J, Osinski C, Li W, Plebanski M, Paulsen H, McKenzie I F: A glycopeptide in complex with MHC class I uses the GalNAc residue as an anchor. Proc Natl Acad Sci USA 2003, 100(25):15029-15034.

22. Apostolopoulos V, Karanikas V, Haurum J S, McKenzie I F: Induction of HLA-A2-restricted CTLs to the Mucin 1 human breast cancer antigen. J Immunol 1997, 159(11):5211-5218.

23. Cibotti R, Kanellopoulos J M, Cabaniols J P, Halle-Panenko O, Kosmatopoulos K, Sercarz E, Kourilsky P: Tolerance to a self-protein involves its immunodominant but does not involve its subdominant determinants. Proc Natl Acad Sci USA 1992, 89(1):416-420.

24. Fedoseyeva E V, Boisgerault F, Anosova N G, Wollish W S, Arlotta P, Jensen P E, Ono S J, Benichou G: CD4+ T cell responses to self- and mutated p53 determinants during tumourigenesis in mice. J Immunol 2000, 164(11):5641-5651.

25. McArdle S E, Rees R C, Mulcahy K A, Saba J, McIntyre C A, Murray A K: Induction of human cytotoxic T lymphocytes that preferentially recognise tumour cells bearing a conformational p53 mutant. Cancer Immunol Immunother 2000, 49(8):417-425.

26. Theobald M, Biggs J, Hernandez J, Lustgarten J, Labadie C, Sherman L A: Tolerance to p53 by A2.1-restricted cytotoxic T lymphocytes. J Exp Med 1997, 185(5):833-841.

27. Andersen M H, Tan L, Sondergaard I, Zeuthen J, Elliott T, Haurum J S: Poor correspondence between predicted and experimental binding of peptides to class I MHC molecules. Tissue Antigens 2000, 55(6):519-531.

28. Apostolopoulos V, McKenzie I F, Pietersz G A: Generation of MUC1 cytotoxic T-cells in mice and epitope mapping. Methods Mol Biol 2000, 125:455-462.

29. Sette A, Vitiello A, Reherman B, Fowler P, Nayersina R, Kast W M, Melief C J, Oseroff C, Yuan L, Ruppert J et al: The relationship between class I binding affinity and immunogenicity of potential cytotoxic T cell epitopes. J Immunol 1994, 153(12):5586-5592.

30. van der Burg S H, Visseren M J, Brandt R M, Kast W M, Melief C J: Immunogenicity of peptides bound to MHC class I molecules depends on the MHC-peptide complex stability. J Immunol 1996, 156(9):3308-3314.

31. Rammensee H G, Friede T, Stevanoviic S: MHC ligands and peptide motifs: first listing. Immunogenetics 1995, 41(4):178-228.

32. Rivoltini L, Squarcina P, Loftus D J, Castelli C, Tarsini P, Mazzocchi A, Rini F, Viggiano V, Belli F, Parmiani G: A superagonist variant of peptide MART1/Melan A27-35 elicits anti-melanoma CD8+ T cells with enhanced functional characteristics: implication for more effective immunotherapy. Cancer Res 1999, 59(2):301-306.

33. Leggatt G R, Hosmalin A, Pendleton C D, Kumar A, Hoffman S, Berzofsky J A: The importance of pairwise interactions between peptide residues in the delineation of TCR specificity. J Immunol 1998, 161(9):4728-4735.

34. Hernandez J, Schoeder K, Blondelle S E, Pons F G, Lone Y C, Simora A, Langlade-Demoyen P, Wilson D B, Zanetti M: Antigenicity and immunogenicity of peptide analogues of a low affinity peptide of the human telomerase reverse transcriptase tumour antigen. Eur J Immunol 2004, 34(8):2331-2341.

35. Okazaki T, Pendleton C D, Lemonnier F, Berzofsky J A: Epitope-enhanced conserved HIV-1 peptide protects HLA-A2-transgenic mice against virus expressing HIV-1 antigen. J Immunol 2003, 171(5):2548-2555.

36. Kuhns J J, Batalia M A, Yan S, Collins E J: Poor binding of a HER-2/neu epitope (GP2) to HLA-A2.1 is due to a lack of interactions with the center of the peptide. J Biol Chem 1999, 274(51):36422-36427.

37. Sharma A K, Kuhns J J, Yan S, Friedline R H, Long B, Tisch R, Collins E J: Class I major histocompatibility complex anchor substitutions alter the conformation of T cell receptor contacts. J Biol Chem 2001, 276(24):21443-21449.

38. Kersh G J, Miley M J, Nelson C A, Grakoui A, Horvath S, Donermeyer D L, Kappler J, Allen P M, Fremont D H: Structural and functional consequences of altering a peptide MHC anchor residue. J Immunol 2001, 166(5):3345-3354.

39. Bownds S, Tong-On P, Rosenberg S A, Parkhurst M: Induction of tumour-reactive cytotoxic T-lymphocytes using a peptide from NY-ESO-1 modified at the carboxy-terminus to enhance HLA-A2.1 binding affinity and stability in solution. J Immunother 2001, 24(1):1-9.

40. Scardino A, Gross D A, Alves P, Schultze J L, Graff-Dubois S, Faure O, Tourdot S, Chouaib S, Nadler L M, Lemonnier F A et al: HER-2/neu and hTERT cryptic epitopes as novel targets for broad spectrum tumour immunotherapy. J Immunol 2002, 168(11):5900-5906.

41. Gross D A, Graff-Dubois S, Opolon P, Cornet S, Alves P, Bennaceur-Griscelli A, Faure O, Guillaume P, Firat H, Chouaib S et al: High vaccination efficiency of low-affinity epitopes in antitumour immunotherapy. J Clin Invest 2004, 113(3):425-433.

42. Tourdot S, Scardino A, Saloustrou E, Gross D A, Pascolo S, Cordopatis P, Lemonnier F A, Kosmatopoulos K. A general strategy to enhance immunogenicity of low-affinity HLA-A2. 1-associated peptides: implication in the identification of cryptic tumour epitopes. Eur J Immunol 2000; 30:3411-21.

43. Tirosh B, el-Shami K, Vaisman N, Carmon L, Bar-Haim E, Vadai E, Feldman M, Fridkin M, Eisenbach L: Immunogenicity of H-2Kb-low affinity, high affinity, and covalently-bound peptides in anti-tumour vaccination. Immunol Lett 1999, 70(1):21-28.

44. Ma H, Kapp J A: Peptide affinity for MHC influences the phenotype of CD8(+) T cells primed in vivo. Cell Immunol 2001, 214(1):89-96.

45. Chang S H, Kim J, Lee K Y, Kim H J, Chung Y J, Park C U, Kim B S, Jang Y S: Modification of the inhibitory amino acid for epitope peptide binding onto major histocompatibility complex class II molecules enhances immunogenicity of the antigen. Scand J Immunol 2004, 59(2):123-132.

46. Fremont D H, Matsumura M, Stura E A, Peterson P A, Wilson I A. Crystal structures of two viral peptides in complex with murine MHC class I H-2Kb. Science 1992; 257:919-27.

47. Jackson M R, Song E S, Yang Y, Peterson P A. Empty and peptide-containing conformers of class I major histocompatibility complex molecules expressed in Drosophila melanogaster cells. Proc Natl Acad Sci USA 1992; 89:12117-21.

48. Saito Y, Peterson P A, Matsumura M. Quantitation of peptide anchor residue contributions to class I major histocompatibility complex molecule binding. J Biol Chem 1993; 268:21309-17.

49. Matsumura M, Saito Y, Jackson M R, Song E S, Peterson P A. In vitro peptide binding to soluble empty class I major histocompatibility complex molecules isolated from transfected Drosophila melanogaster cells. J Biol Chem 1992; 267:23589-95.

50. Apostolopoulos V, Pouniotis D S, van Maanen P J et al. Delivery of tumor associated antigens to antigen presenting cells using penetratin induces potent immune responses. Vaccine 2006; 24(16):3191-202.

51. Plebanski M, Burtles S S. In vitro primary responses of human T cells to soluble protein antigens. J Immunol Methods 1994; 170:15-25.

52. Plebanski M, Gilbert S C, Schneider J et al. Protection from Plasmodium berghei infection by priming and boosting T cells to a single class I-restricted epitope with recombinant carriers suitable for human use. Eur J Immunol 1998; 28:4345-55.

53. Pietersz G A, Li W, Popovski V, Caruana J A, Apostolopoulos V, McKenzie I F. Parameters for using mannan-MUC1 fusion protein to induce cellular immunity. Cancer Immunol Immunother 1998; 45:321-6.

54. Jager E, Jager D, Knuth A. Clinical cancer vaccine trials. Curr Opin Immunol 2002; 14:178-82.

55. Lee E A, Palmer D R, Flanagan K L et al. Induction of T helper type 1 and 2 responses to 19-kilodalton merozoite surface protein 1 in vaccinated healthy volunteers and adults naturally exposed to malaria. Infect Immun 2002; 70:1417-21.

56. Bredenbeck A, Losch F O, Sharav T et al. Identification of noncanonical melanoma-associated T cell epitopes for cancer immunotherapy. J Immunol 2005; 174:6716-24.

57. Lazoura E, Apostolopoulos V: Insights into peptide-based vaccine design for cancer immunotherapy. Current Medicinal Chemistry 2005; 12, 1481-1494.

58. Alexander, J et al, Linear PADRE T Helper Epitope and Carbohydrate B Cell Epitope Conjugates Induce Specific High Titer IgG Antibody Responses. J Immunol 2000; 164:1625.

59. WO 97/11715—International Patent Application entitled “Mimicking Peptides in Cancer Therapy” (PCT/AU96/00617).

60. Kirshenbaum K, Zuckermann R N, Dill K A, Designing polymers that mimic biomolecules. Curr Opin Struct Biol 1999; 9(4):530-5.

61. WO 95/18145—International Patent Application entitled “Conjugates of Human Mucin and a Carbohydrate Polymer and their use in cancer Treatment” (PCT/AU94/00789).

62. Sambrook et al, Molecular Cloning: A Laboratory Manual, Coldspring Harbour Laboratory Press, NY, second edition, 1989.

63. Loveland B E, et al., Mannan-MUC1-Pulsed Dendritic Cell Immunotherapy: A Phase I Trial in Patients with Adenocarcinoma. Clin Cancer Res 2006; 12(3):869-877.

1. A vaccine for the prevention or treatment of cancer, said vaccine comprising at least one Mucin 1 (MUC1) T cell epitope-derived peptide or peptide analogue optionally conjugated to at least one helper molecule that binds to human leukocyte antigen (HLA) class II protein, such that the vaccine, upon administration to a subject, elicits a cytotoxic T cell (CTL) response to Mucin 1.

2. The vaccine of claim 1, wherein the at least one HLA class II protein-binding helper molecule binds to two or more HLA class II protein types or haplotypes.

3. The vaccine of claim 1, wherein the MUC1 T cell epitope-derived peptide or peptide analogue is a peptide comprising an amino acid sequence corresponding to one that is native to mice or one that is native to humans but modified inasmuch as the amino acid sequence of the peptide incorporates one or more amino acid substitutions.

4. The vaccine of claim 3, wherein said one or more amino acid substitutions are located at one or more of the non-anchor residue positions of the relevant native MUC1 T cell epitope.

5. The vaccine of claim 3, wherein said one or more amino acid substitutions are located at one or more non-anchor residue positions and one or more anchor residue positions.

6. The vaccine of claim 3, wherein the peptide is a 9-mer.

7. The vaccine of claim 3, wherein the MUC1 T cell epitope-derived peptides are derived from a native mouse or a native human MUC1 T cell epitope selected from:

| | |
|---|---|
| (i) STAPPAHGV; | (SEQ ID NO: 5) |
| and | |
| (ii) SAPDTRPAP. | (SEQ ID NO: 8) |

8. The vaccine of claim 3, wherein the MUC1 T cell epitope-derived peptide is according to formula (I):

| | |
|---|---|
| (I) X^{a}-Xⁱ-TAPP-X⁶-HG-X⁹-X^{b}; | (SEQ ID NO: 9) |

wherein:
X^{a} is absent or any amino acid or sequence of any two to five amino acids,
X¹ is selected from Ser (S) and Thr (T), X⁶ is selected from Ala (A), Val (V), Leu (L) and Ile (I),
X⁹ is absent or selected from Val (V), Leu (L), Ile (I), Met (M), Phe (F), Ala (A) and Nle, and
X^{b} is absent or any amino acid or sequence of any two to five amino acids.

9. The vaccine of claim 8, wherein X^{a} is absent, X¹ is selected from S and T, X⁶ is selected from A and V, X⁹ is selected from V and L, and X^{b} is absent.

10. The vaccine of claim 3, wherein the MUC1 T cell epitope-derived peptide consists of one of the following amino acid sequences:

| | |
|---|---|
| (i) TTAPPVHGL; | (SEQ ID NO: 6) |
| (ii) STAPPVHGL; | (SEQ ID NO: 10) |
| (iii) STAPPAHGL; | (SEQ ID NO: 11) |
| (iv) TTAPPAHGV; | (SEQ ID NO: 12) |
| and | |
| (v) SAPDTYPAL. | (SEQ ID NO: 13) |

11. The vaccine of claim 1, wherein the MUC1 T cell epitope-derived peptide or peptide analogue is an analogue of a peptide according to formula (I):

| | |
|---|---|
| (I) X^{a}-Xⁱ-TAPP-X⁶-HG-X⁹-X^{b}; | (SEQ ID NO: 9) |

wherein:
X^{a} is absent or any amino acid or sequence of any two to five amino acids,
X¹ is selected from Ser (S) and Thr (T),
X⁶ is selected from Ala (A), Val (V), Leu (L) and Ile (I),
X⁹ is absent or selected from Val (V), Leu (L), Ile (I), Met (M), Phe (F), Ala (A) and Nle, and
X^{b} is absent or any amino acid or sequence of any two to five amino acids.

12. The vaccine of claim 1, wherein the the MUC1 T cell epitope-derived peptide or peptide analogue is an analogue of a peptide consisting of one of the following amino acid sequences:

| | |
|---|---|
| (i) TTAPPVHGL; | (SEQ ID NO: 6) |
| (ii) STAPPVHGL; | (SEQ ID NO: 10) |
| (iii) STAPPAHGL; | (SEQ ID NO: 11) |
| (iv) TTAPPAHGV; | (SEQ ID NO: 12) |
| and | |
| (v) SAPDTYPAL. | (SEQ ID NO: 13) |

13. The vaccine of claim 1, wherein the MUC1 T cell epitope-derived peptide or peptide analogue is conjugated to at least one helper molecule that binds to HLA class II protein.

14. The vaccine of claim 13, wherein the at least one HLA class II protein-binding helper molecule is selected from the group consisting of keyhole limpet haemocyanin (KLH), tetanus toxoid (TT), diphtheria toxoid, PADRE peptides, and combinations thereof.

15. The vaccine of claim 14, wherein the at least one HLA class II protein-binding helper molecule is KLH.

16. The vaccine of claim 1, further comprising a pharmaceutically acceptable carrier.

17. The vaccine of claim 16, wherein the pharmaceutically acceptable carrier is a carrier molecule selected from the group consisting of mannan, oxidised mannan, partially oxidised mannan, reduced mannan, the TAT protein from human immunodeficiency virus (HIV), the VP22 protein from herpes simplex virus (HSV), the amphipathic peptide Pep-1, the 60 amino acid DNA binding domain of the Drosophila melanogaster transcription factor, Antennapedia, the 16 amino acid region of Antennapedia responsible for cellular internalisation and other receptor-mediated carrier molecules.

18. The vaccine of claim 17, wherein the carrier molecule is oxidised mannan.

19. A method of prevention or treatment of cancer in a subject, said method comprising administering to said subject an effective amount of the vaccine of claim 1.

20. A composition for the ex vivo priming of dendritic cells (DCs), said composition comprising at least one Mucin 1 (MUC1) T cell epitope-derived peptide or peptide analogue optionally conjugated to at least one helper molecule that binds to human leukocyte antigen (HLA) class II protein.

21. The composition of claim 20, wherein the at least one HLA class protein-binding helper molecule binds to two or more HLA class II protein types or haplotypes.

22. The composition of claim 20, wherein the MUC1 T cell epitope-derived peptide or peptide analogue is a peptide comprising an amino acid sequence corresponding to one that is native to mice or one that is native to humans but modified inasmuch as the amino acid sequence of the peptide incorporates one or more amino acid substitutions.

23. The composition of claim 22, wherein said one or more amino acid substitutions are located at one or more of the non-anchor residue positions of the relevant native MUC1 T cell epitope.

24. The composition of claim 22, wherein said one or more amino acid substitutions are located at one or more non-anchor residue positions and one or more anchor residue positions.

25. The composition of claim 22, wherein the peptide is a 9-mer.

26. The composition of claim 22, wherein the MUC1 T cell epitope-derived peptides are derived from a native mouse or a native human MUC1 T cell epitope selected from:

| | |
|---|---|
| (i) STAPPAHGV; | (SEQ ID NO: 5) |
| and | |
| (ii) SAPDTRPAP. | (SEQ ID NO: 8) |

27. The composition of claim 22, wherein the MUC1 T cell epitope-derived peptide is according to formula (I):

| | |
|---|---|
| (I) X^{a}-Xⁱ-TAPP-X⁶-HG-X⁹-X^{b}; | (SEQ ID NO: 9) |

wherein:
X^{a} is absent or any amino acid or sequence of any two to five amino acids,
Xⁱ is selected from Ser (S) and Thr (T),
X⁶ is selected from Ala (A), Val (V), Leu (L) and Ile (I),
X⁹ is absent or selected from Val (V), Leu (L), Ile (I), Met (M), Phe (F), Ala (A) and Nle, and
X^{b} is absent or any amino acid or sequence of any two to five amino acids.

28. The composition of claim 27, wherein X^{a} is absent, X¹ is selected from S and T, X⁶ is selected from A and V, X⁹ is selected from V and L, and X^{b} is absent.

29. The composition of claim 22, wherein the MUC1 T cell epitope-derived peptide consists of one of the following amino acid sequences:

| | |
|---|---|
| (i) TTAPPVHGL; | (SEQ ID NO: 6) |
| (ii) STAPPVHGL; | (SEQ ID NO: 10) |
| (iii) STAPPAHGL; | (SEQ ID NO: 11) |
| (iv) TTAPPAHGV; | (SEQ ID NO: 12) |
| and | |
| (v) SAPDTYPAL. | (SEQ ID NO: 13) |

30. The composition of claim 20, wherein the MUC1 T cell epitope-derived peptide or peptide analogue is an analogue of a peptide according to formula (I):

| | |
|---|---|
| (I) X^{a}-Xⁱ-TAPP-X⁶-HG-X⁹-X^{b}; | (SEQ ID NO: 9) |

wherein:
X^{a} is absent or any amino acid or sequence of any two to five amino acids,
X¹ is selected from Ser (S) and Thr (T),
X⁶ is selected from Ala (A), Val (V), Leu (L) and Ile (I),
X⁹ is absent or selected from Val (V), Leu (L), Ile (I), Met (M), Phe (F), Ala (A) and Nle, and
X^{b} is absent or any amino acid or sequence of any two to five amino acids.

31. The composition of claim 20, wherein the MUC1 T cell epitope-derived peptide or peptide analogue is an analogue of a peptide consisting of one of the following amino acid sequences:

| | |
|---|---|
| (i) TTAPPVHGL; | (SEQ ID NO: 6) |
| (ii) STAPPVHGL; | (SEQ ID NO: 10) |
| (iii) STAPPAHGL; | (SEQ ID NO: 11) |
| (iv) TTAPPAHGV; | (SEQ ID NO: 12) |
| and | |
| (v) SAPDTYPAL. | (SEQ ID NO: 13) |

32. The composition of claim 20, wherein the MUC1 T cell epitope-derived peptide or peptide analogue is conjugated to at least one helper molecule that binds to HLA class II protein.

33. The composition of claim 32, wherein the at least one HLA class II protein-binding helper molecule is selected from the group consisting of keyhole limpet haemocyanin (KLH), tetanus toxoid (TT), diphtheria toxoid, PADRE peptides, and combinations thereof.

34. The composition of claim 33, wherein the at least one HLA class II protein-binding helper molecule is KLH.

35. The composition of claim 20, further comprising a pharmaceutically acceptable carrier.

36. The composition of claim 35, wherein the pharmaceutically acceptable carrier is a carrier molecule selected from the group consisting of mannan, oxidised mannan, partially oxidised mannan, reduced mannan, the TAT protein from human immunodeficiency virus (HIV), the VP22 protein from herpes simplex virus (HSV), the amphipathic peptide Pep-1, the 60 amino acid DNA binding domain of the Drosophila melanogaster transcription factor, Antennapedia, the 16 amino acid region of Antennapedia responsible for cellular internalisation and other receptor-mediated carrier molecules.

37. The composition of claim 36, wherein the carrier molecule is oxidised mannan.

38. A method of prevention or treatment of cancer in a subject, said method comprising the steps of treating dendritic cells (DCs) ex vivo with the composition of claim 20, such that the DCs are primed to MUC1, and thereafter administering the primed DCs to said subject.

39. The method of claim 38, wherein the step of treating the DCs with the composition ex vivo is achieved by pulsing the DCs in the presence of said composition.

40. A MUC1 T cell epitope-derived peptide consisting of one of the following amino acid sequences:

| | |
|---|---|
| (i) TTAPPVHGL; | (SEQ ID NO: 6) |
| (ii) STAPPVHGL; | (SEQ ID NO: 10) |
| (iii) STAPPAHGL; | (SEQ ID NO: 11) |
| (iv) TTAPPAHGV; | (SEQ ID NO: 12) |
| and | |
| (v) SAPDTYPAL, | (SEQ ID NO: 13) |
| in a substantially purified form. | |

41. A fusion polypeptide comprising a MUC1 T cell epitope-derived peptide consisting of one of the following amino acid sequences:
(i) TTAPPVHGL (SEQ ID NO: 6);
(ii) STAPPVHGL (SEQ ID NO: 10);
(iii) STAPPAHGL (SEQ ID NO: 11);
(iv) TTAPPAHGV (SEQ ID NO: 12); and
(v) SAPDTYPAL (SEQ ID NO: 13), fused to a helper molecule that binds to human leukocyte antigen (HLA) class II protein.

42. The fusion polypeptide of claim 41, wherein the HLA class II protein-binding helper molecule is keyhole limpet haemocyanin (KLH).

43. A polynucleotide molecule comprising a nucleotide sequence encoding the fusion polypeptide of claim 41.

44. The vaccine of claim 3, wherein said one or more amino acid substitutions are located at one or more of the anchor residue positions of the relevant native MUC1 T cell epitope.

45. The composition of claim 22, wherein said one or more amino acid substitutions are located at one or more of the anchor residue positions of the relevant native MUC1 T cell epitope.
